(19) United States
(12) Patent Application Publication
(10) Pub. No.: US 2026/0184672 A1
(43) Pub. Date: Jul. 2, 2026

(54) MACROCYCLIC SUPRAMOLECULAR POLYMERS

(71) Applicant: The Trustees of Indiana University, Bloomington, IN (US)

(72) Inventors: Amar H. Flood, Bloomington, IN (US); Yusheng Chen, Bloomington, IN (US)

(21) Appl. No.: 19/420,284

(22) Filed: Dec. 15, 2025

Related U.S. Application Data
(60) Provisional application No. 63/734,043, filed on Dec. 14, 2024.

(51) Int. Cl. C07C 261/02; C07F 9/11; C07F 9/12

(52) U.S. Cl. CPC C07C 261/02; C07F 9/11; C07F 9/12

(57) ABSTRACT

A supramolecular material comprising a 2:2:2 assembly, the 2:2:2 assembly comprising a pair of macrocycles, two anionic species dimerized within the pair of macrocycles, each anionic species is a conjugate base of an organophosphoric acid moiety or a conjugate base of an organophosphonic acid moiety, and two cationic species, wherein each of the cationic species comprise a protonated nitrogen and each of the cationic species are hydrogen bonded by a N⁺—H···O salt bridge to an oxygen of the anionic species. Supramolecular polymers comprising a plurality of 2:2:2 assemblies are also provided. Methods for preparing the supramolecular materials are also provided.

### CROSS-REFERENCE TO RELATED APPLICATIONS

The present application claims priority to U. S. Provisional Patent Application No. 63/734,043 that was filed Dec. 14, 2024, the entire contents of which are hereby incorporated by reference.

### STATEMENT REGARDING FEDERALLY SPONSORED RESEARCH OR DEVELOPMENT

This invention was made with government support under 2105848 awarded by National Science Foundation. The Government has certain rights in the invention.

### BACKGROUND OF THE INVENTION

Cations are emerging as functional and active partners in the hierarchical assembly of anion-driven architectures. Examples include use of cations as templates in phosphate-driven cages, fluorescent cations in optical materials, and as structural partners in chemically-driven crystallization. At the heart of these assemblies is a receptor-anion complex. They serve as the primary structure upon which higher levels of structural order are layered. Charge-balancing organic and inorganic cations are a potential source of higher order. Some studies use quaternary (4°) ammonium as inert cations of which tetra-n-butylammonium cation (TBA+) is common and exemplary. The N-substitution in the tetra-n-butylammonium cation with four alkyl chains turns off both specific interactions, e.g., H-bonds, and structural ordering to help disfavor ion pairing and liberate the anions for binding. While this approach has been successful for the purposes of binding anions, it has come at the expense of diversity and structural ordering.

Building upon these insights the assembly of receptors, anions and cations, researchers have begun to explore how such interactions can be extended into larger, more complex frameworks. The principles of charge balance, structural organization, and controlled interactions provide a foundation for designing dynamic systems that go beyond discrete cages or crystalline materials to polymers. Despite great scientific advances in the field of supramolecular polymers, polymer materials that provide properties of being reversible, reconfigurable, healable, and environmentally responsive remain of high value. Accordingly, there is a need in the art for materials that will be remoldable, self-adaptive, and recyclable.

### BRIEF SUMMARY OF THE INVENTION

The present invention provides supramolecular materials and polymers that offer advantages including reversibility, reconfigurability, healability, and environmental responsiveness. The materials may be remoldable, self-adaptive, and recyclable. The disclosed technology enables straightforward preparation through simple mixing of components without requiring heating, and provides access to diverse material properties including gels, elastomers, adhesives, and fibers through selection of starting materials.

In one aspect, the technology relates to a supramolecular material comprising a 2:2:2 assembly, the 2:2:2 assembly comprising a pair of macrocycles, two anionic species dimerized within the pair of macrocycles, each anionic species being a conjugate base of an organophosphoric acid moiety or a conjugate base of an organophosphonic acid moiety, and two cationic species, wherein each of the cationic species comprise a protonated nitrogen and each of the cationic species are hydrogen bonded by a N⁺—H···O salt bridge to an oxygen of the anionic species.

In another aspect, the technology relates to a supramolecular polymer comprising a plurality of 2:2:2 assemblies. The supramolecular polymer may comprise a plurality of compounds having at least one tertiary ammonium moiety or at least one protonated nitrogen-containing heteroaryl moiety. In some aspects, the supramolecular polymer may be a linear polymer or a network polymer.

In yet another aspect, the technology relates to a method for preparing a material, the method comprising mixing macrocycles, first compounds having at least one tertiary amine moiety or at least one nitrogen-containing heteroaryl moiety, and second compounds having at least one phosphoric acid moiety or at least one phosphonic acid moiety, wherein the mixing is performed under conditions suitable for proton transfer from second compounds to first compounds and formation of N⁺—H···O salt bridges between first compounds and second compounds.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows: formation of hierarchical ionic assembly by leveraging proton transfer between acid and amine for forming salt bridges. (a) Crystal structure of cyanostar, phenyl phosphate and 4° TBA⁺ cations (CCDC #2312047) providing inspiration for (b) replacing 4° cations with H-bonding 3° cations to form salt bridges. (c) Cyanostar mixed with phenyl phosphoric acid and dicyclohexyl-methyl amine undergoes (d) proton transfer to form the anion and cation capable of hierarchical formation of a 2:2:2 assembly (CCDC #2312046) showing (1) NH, (1) OH and (3) CH H-bonding and (e) the salt bridge, 2:2:2 hierarchical ionic assembly, and anion stabilization.

FIG. 2 shows the diverse N-bases used in this work. Variety of 19 amines and N-heterocyclic bases tested in this study to explore a diverse set of building blocks. The pKₐ values are listed and yields of co-assembly with phenyl-phosphate anion and the cyanostar are listed as percentages.

FIG. 3 shows NMR characterization of 2:2:2 assembly. ¹H NMR spectra of 2:2:2 ternary assembly formed from an equimolar mixture of phenyl phosphoric acid, dicyclohexyl-methyl amine, and cyanostar. DOSY data of the ternary assembly. (10 mM, CD₂Cl₂, 600 MHz, 298 K).

FIG. 4 shows equilibrium between reactants and the 2:2:2 assembly products. (a) ¹H NMR titration of the ion pair formed between triisopentyl ammonium and 4-trifluoromethylphenyl vinyl phosphonate into cyanostar (1 mM, CD₂Cl₂, 298 K, 600 MHz). Black solid circles are cyanostar dimers, empty circles are the free cyanostar. Black solid star indicates OH H-bond, black empty stars indicate signals of excess phosphonate. Black solid triangles indicate NH H-bond in 2:2:2 assembly, the empty black triangle indicates H-bond in ion pair. (b) Equilibrium between reactants composed of the ammonium-phosphonate ion pair and free cyanostar and the 2:2:2 assembly products.

FIG. 5 shows high-throughput experimentation using 72 different combinations of acids and amines. (a) Amines and N-heterocyclic bases listed A to I and (b) acids numbered 1 to 8. (c) The black boxes indicates 100% yield of ternary assembly, other gradations of grey indicate varying degrees of assembly (16-74%), the a 100 indicates 100% free cyanostar and the boxes with a slash through them indicates precipitation or ambiguous NMR spectra. All samples at 1.6 mM in dichloromethane (1-4) or 54:44 dichloromethane-acetonitrile (5-8). (d) Combination of the 4° TBA⁺ cation with anions 1-8.

FIG. 6 shows the crystal structure of the 2:2:2 assembly consisting of 1-methyl imidazolium, phenyl phosphate and cyanostar (CCDC #2352315). Dashed lines indicate OH and NH hydrogen bonds.

FIG. 7 shows schematic energy profile for assembly. Curves show estimates of the potential wells that describe the assembly of cyanostar with combinations of different organophosphate anions and nitrogen-based cations. These are compared to the estimated curve for assembly with TBA-phenyl phosphate. The reaction free energies (ΔGᵣₓₙ/kJ mol⁻¹) are listed for B1 and C2 while relative ordering is provided for F3 and H2.

FIG. 8 shows a list of organophosphoric acids. Summary of different acids used in high-throughput experiments, 1: dodecyl dihydrogen phosphate; 2: phenylphosphoric acid; 3: 1-naphthyl dihydrogen phosphate; 4: triethylene glycol bis(phenyl-p-phosphoric acid); 5: benzylphosphonic acid; 6: trifluoromethylphenyl vinyl phosphonic acid; 7: phenylphosphonic acid; 8: di(2-ethylhexyl) fluorene bis(vinylphosphonic acid).

FIG. 9 shows a list of amines. Summary of different amines used in high-throughput experiments, A: N,N-Diisopropyl-3-pentylamine; B: tribenzylamine; C: dicyclohexylmethylamine; D: triisopentylamine; E: N1,N1,N6,N6-tetramethylhexane-1,6-diamine; F: 4-(tert-butyl)pyridine; G: 4-methylquinoline; H: 9-phenylacridine; I: 4-(trifluoromethyl)pyridine.

FIG. 10 shows: (a) Previous work of supramolecular polymerization based on anion-anion dimerization. (b) 2:2:2 assembly based on O—H···O, N⁺—H···O and C—H···O hydrogen bonds. (c) Supramolecular polymerization using diamine, mono-phosphoric acid, cyanostar and (d) generation of supramolecular networks using diamine, di-phosphoric acid and cyanostar.

FIG. 11 shows: (a) The macroscopic change of supramolecular polymerization using diamine Et₂N—C₁₂H₂₄—NEt₂, phenyl phosphoric acid and cyanostar (3 mM, CH₂Cl₂, 298 K). (b) Proposed progress in microscopic scale based on the SEM image.

FIG. 12 shows: (a) A mixture of the diammonium, mono-phosphate and cyanostar used to generate a supramolecular polymer as seen in the (b) crystal structure. (c) Repeating chemical structure of the anions and cations forming the backbone of the supramolecular polymer.

FIG. 13 shows: (a) Synthetic route of diamines with different lengths. (b) Stack of ¹H NMR spectra of stoichiometric mixture of phenylphosphoric acid, cyanostar and diamines with different length (1 mM/CD₂Cl₂/298 K/600 MHz). Spectra show a mixture of free cyanostar signals and signals of the cyanostar dimer in the supramolecular polymer change with chain length. (c) The plot of dimerization percentage versus number of atoms between two amine end-groups and model of self-associated cyclic pseudo[3]catenane.

FIG. 14 shows: (a) Synthesis of 2D supramolecular networks by mixing stoichiometric amount of ditopic phenylphosphoric acid, diamine and cyanostar. (b) ¹H NMR spectrum of supramolecular network, squares indicate defects or oligomers (10 mM cyanostar/CD₂Cl₂/298 K/600 MHz). (c) Diffusion NMR of supramolecular networks (10 mM cyanostar/CD₂Cl₂/298 K/600 MHz).

FIG. 15 shows: (a) Chemical components in a 1D supramolecular polymer and (b-e) SEM images of 3 mM supramolecular polymer suspension. (f) Chemical components in a 2D supramolecular network and SEM images of (g-h) 3 mM 2D network solution and (i-j) 10 mM network prepared as suspension in dichloromethane.

### DETAILED DESCRIPTION OF THE INVENTION

The present disclosure is described herein using several definitions, as set forth below and throughout the application.

The present disclosure provides a supramolecular material comprising a 2:2:2 assembly. The 2:2:2 assembly comprises a pair of macrocycles, two anionic species dimerized within the pair of macrocycles, each anionic species being a conjugate base of an organophosphoric acid moiety or a conjugate base of an organophosphonic acid moiety, and two cationic species, wherein each of the cationic species comprise a protonated nitrogen and each of the cationic species are hydrogen bonded by a N⁺—H···O salt bridge to an oxygen of the anionic species. The combination of the macrocycles, the dimerized anionic species, and the cationic species results in a supramolecular material stabilized by a network of non-covalent interactions. The macrocycles encapsulate the anionic species, while the cationic species provide external stabilization through hydrogen bonding. The assembly is driven by complementary electrostatics, hydrogen bonding, and cavity confinement, to yield the supramolecular complex.

As used herein, the term macrocycle refers to a cyclic organic molecule having a cavity or binding pocket capable of accommodating guest species through non-covalent interactions. In certain embodiments, the macrocycle is a polycyanostilbene macrocycle and an electron-rich interior cavity. Polycyanostilbene may be characterized by a fivefold symmetric alternating arrangement of five phenyl rings and cynaosubstituted ethenylene moieties linking the phenyl rings. The phenyl ring may be substituted with a variety of different substituents. Exemplary phenyl substituents include alkyl, alkenyl, alkynyl, alkoxyl, cycloalkyl, heterocyclyl, aryl, heteroaryl, amino, or halo. Examples of polycyanostilbenes are disclosed in U.S. Pat. No. 9,701,621, which is incorporated by reference in its entirety. In some embodiments, the macrocycle is

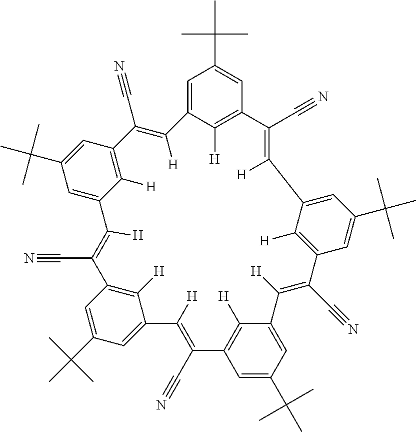 [SMILES: CC(C)(C)c1cc2cc(c1)/C(C#N)=C/c1cc(cc(C(C)(C)C)c1)/C(C#N)=C/c1cc(cc(C(C)(C)C)c1)/C(C#N)=C/c1cc(cc(C(C)(C)C)c1)/C(C#N)=C/c1cc(cc(C(C)(C)C)c1)/C(C#N)=C/2]

that may be referred to as cyanostar. The pair of macrocycles may associate in a face-to-face arrangement.

The supramolecular material further comprises two anionic species, each being a conjugate base of an organophosphoric acid moiety or a conjugate base of an organophosphonic acid moiety. Upon proton transfer to a tertiary amine moiety or nitrogen-containing heteroaryl, the organophosphoric or organophosphonic acid generates a conjugate base (i.e., a phosphate moiety or phosphonate moiety) that exists as an anion. These anion species dimerize within the cavity formed by the pair of macrocycles. Compounds may comprise 1, 2, 3, or more than 3 phosphoric acid or phosphonic acid moieties that can result in compounds having 1, 2, 3, or more than 3 phosphate or phosphonate moieties, respectively. In some embodiments, the anionic species is derived from a diacid. In some embodiments, the anionic species is derived from a compound selected from the group consisting of:

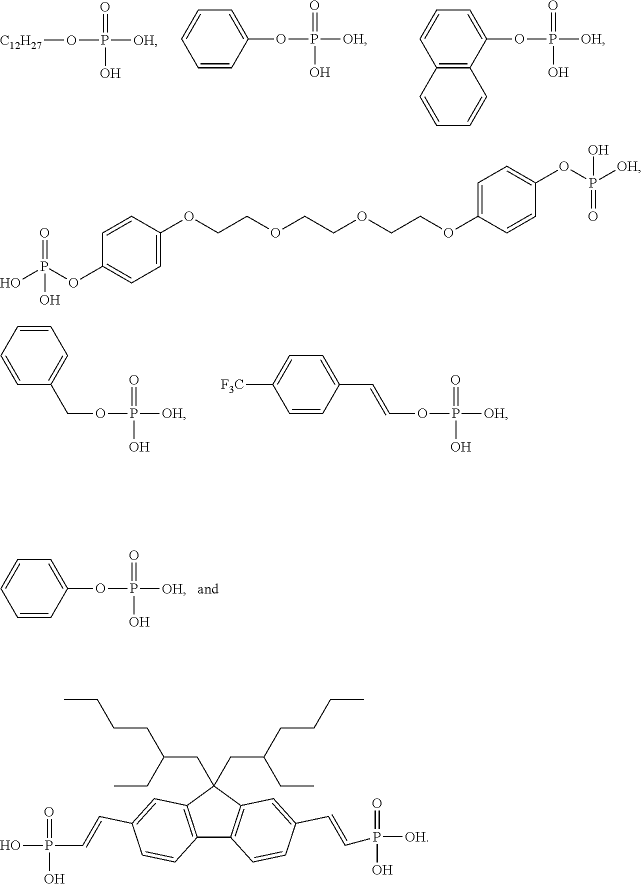

In some embodiments, each of the two cationic species comprise a protonated nitrogen. In some embodiments, the cationic species is a tertiary ammonium moiety. In some embodiments, the cationic species is a protonated nitrogen-containing heteroaryl moiety. Compounds may comprise 1, 2, 3, or more than 3 tertiary amine moieties or nitrogen-containing heteroaryl that can result in 1, 2, 3, or more 3 ammonium moieties or protonated heteroaryl moieties. Upon proton transfer from the organophosphoric or organophosphonic acid, the moiety, e.g., a tertiary amine, is converted into its conjugate acid, existing as a cation. In some embodiments, the cationic species are derived from the group consisting of N,N-diisopropylpentan-3-amine, tribenzylamine, N-cyclohexyl-N-methylcyclohexanamine, triisopentylamine, 4-(tert-butyl)pyridine, 4-methylquinoline, 9-phenylacridine, 4-(trifluoromethyl)pyridine, N¹,N¹,N⁶,N⁶-tetramethylhexane-1,6-diamine, N¹,N¹,N²,N²-tetraethyldodecane-1,12-diamine, 3,3′-(1,4-phenylenebis(oxy))bis(N,N-diethylpropan-1-amine), 6,6′-(1,4-phenylenebis(oxy))bis(N,N-diethylhexan-1-amine), 10,10′-(1,4-phenylenebis(oxy))bis(N,N-diethyldecan-1-amine), 10,10′-(1,4-phenylenebis(oxy))bis(N,N-dimethyldecan-1-amine), 14,14′-(1,4-phenylenebis(oxy))bis(N,N-diethyltetradecan-1-amine), and 2,2′-((oxybis(ethane-2,1-diyl))bis(oxy))bis(N,N-diethylethan-1-amine). In some embodiments, the cationic species is a pyridinium moiety, a quinolinium moiety, an acridinium moiety, or an imidazolium moiety. The choice of amine influences the strength of proton transfer, and suitable amines are selected such that the pKa difference between the acid and the amine exceeds three (3) units to ensure proton transfer and formation of the cationic and anionic partners.

In some embodiments, the supramolecular material is a supramolecular polymer comprising a plurality of 2:2:2 assemblies. The supramolecular polymer may comprise a plurality of compounds having at least one tertiary ammonium moiety or at least one protonated nitrogen-containing heteroaryl moiety. The supramolecular polymer may be a linear polymer or a network polymer. In some embodiments, the compounds containing tertiary amine moieties or nitrogen-containing heteroaryl moieties can be monotopic, ditopic, or polytopic, e.g., having one, two, three, or more than three moieties that can be protonated and thereby form a cationic species. In some embodiments, the plurality of compounds comprise at least two tertiary ammonium moieties. In some embodiments, the acids can be monotopic, ditopic, or polytopic, e.g., having one, two, three, or more than three phosphoric acid or phosphonic acid moieties that can be deprotonated and thereby form anionic species. In some embodiments, the supramolecular polymer comprises a plurality of compounds having at least one phosphate moiety or at least one phosphonate moiety. In some embodiments, the supramolecular polymer comprises a plurality of compounds having at least two phosphate moieties or at least two phosphonate moieties. One or more of the amines or acids used in supramolecular polymer has to be ditopic or polytopic. The supramolecular polymer cannot be made solely from monotopic acids and monotopic amines.

The polymers described herein are made from three components: macrocycles (such as cyanostars), tertiary amine or nitrogen containing heteroaryl containing compounds, and either organo-phosphoric acids or organo-phosphonic acids. At least one of the nitrogen-containing compounds or and acid compounds has two (or more) acid or two (or more) nitrogen-containing functional groups. The use of the 3° amines in the preparation is of particular interest. Access to over 3000 commercial 3° amines allows the materials properties to be varied systematically to produce polymers with a variety of base mechanical properties, e.g., gels, elastomers, adhesives, fibers. Being supramolecular polymers held together by dynamic non-covalent bonds, the materials may also be remoldable, self-adaptive and recyclable, e.g., reversible glue. In some embodiments, the composition is used to make glues/adhesives that can be reversibly made, broken and remade again like post-it notes. In some embodiments, these materials are made by mixing the components together. No heating is required to form the supramolecular assemblies, but may be optionally provided.

In some embodiments, the polymer materials made provide the property of being reversible, such as reconfigurable, healable, and environmentally responsive. Thus, the material may be remoldable, self-adaptive and recyclable. The polymer can be made into different types of materials. Depending on selection of the starting materials, they can be made into gels, elastomers, adhesives, fibers, etc.

The present disclosure also provides a method for preparing a material. The method comprises mixing macrocycles, first compounds having at least one tertiary amine moiety or at least one nitrogen-containing heteroaryl moiety, and second compounds having at least one phosphoric acid moiety or at least one phosphonic acid moiety, wherein the mixing is performed under conditions suitable for proton transfer from second compounds to first compounds and formation of N⁺—H···O salt bridges between first compounds and second compounds. In some embodiments, the macrocycles are added to a mixture of first compounds and second compounds. Exemplary supramolecular polymers may be prepared from first compounds comprising at least two tertiary ammonium moieties.

Solvents may be selected to provide conditions suitable for proton transfer and formation of the salt bridges. A number of organic solvents be utilized, such as halogenated alkanes (e.g., dichloromethane) or nitriles (e.g., acetonitrile).

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the disclosed subject matter and does not pose a limitation on the scope of the disclosed subject matter unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Aspects of this disclosure are described herein, including the best mode known to the inventors for carrying out the disclosed subject matter. Variations of those aspects may become apparent to those of ordinary skill in the art upon reading the foregoing description. The inventors expect a person having ordinary skill in the art to employ such variations as appropriate, and the inventors intend for the disclosed subject matter to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

In the foregoing description, it will be readily apparent to one skilled in the art that varying substitutions and modifications may be made to the disclosed subject matter without departing from the scope and spirit of the disclosure. The subject matter illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosed subject matter. Thus, it should be understood that although the present disclosure has been illustrated by specific embodiments and optional features, modification and/or variation of the concepts herein disclosed may be resorted to by those skilled in the art, and that such modifications and variations are considered to be within the scope of this disclosure.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

### EXAMPLES

### Example 1

The following Examples are illustrative and should not be interpreted to limit the scope of the claimed subject matter.

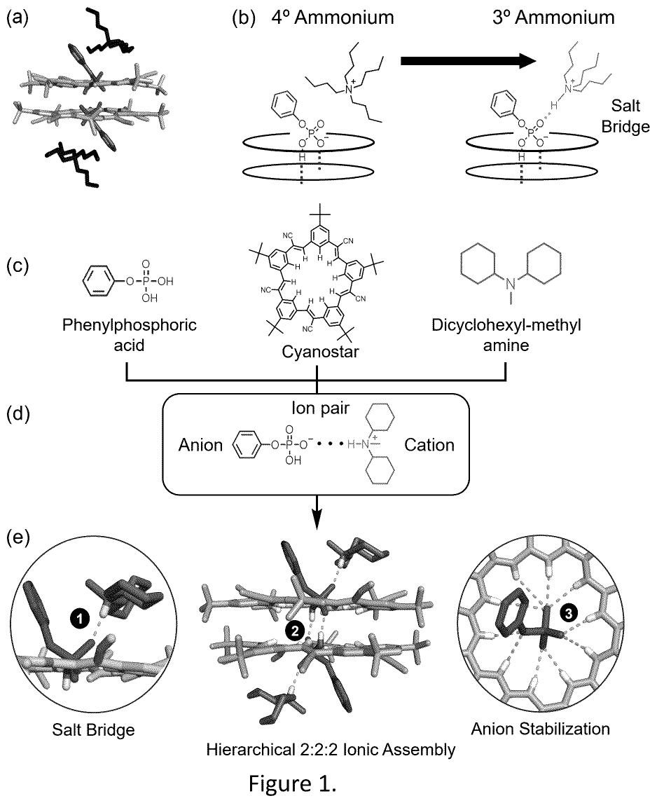

Herein tertiary (3°) ammonium cations that are easy to make using acid-base chemistry are explored. This strategy also turns on directional H-bonding for salt bridge formation in the assembly (FIG. 1b),^{35,36} which is common in solids³⁷⁻⁴⁰ but rare⁴¹ in solution. The H-bonding also turns on ion pairing in the building blocks (FIG. 1d). It is not clear, therefore, whether the NH H-bonds will favor or disfavor product assembly. These ideas are explored by docking 3° ammonium cations with privileged 2:2 complexes⁴² formed between cyanostar macrocycles and organo-phosphate/-phosphonate anions. The 2:2 complex is stable enough to support salt bridges with the cations, and the yield of assembly is controlled by solvent and sterics. Mixing bases, acids, and cyanostar allows formation of 2:2:2 assemblies (FIG. 1c-e), and the 82 examples explored herein opens access to a new layer of diversity and structural ordering in hierarchical ionic assembly.

Use of 3° ammonium cations offers simplicity and scope to hierarchical anion assembly. The cations and associated anions can be made by proton transfer^{43,44} upon adding 3° amines to acids with pKa differences exceeding 4.45 E.g., mixing 3° amines (pKa˜11) with phenyl phosphoric acid (pKa 1˜2) allows preparation of the ionic assembly partners. Diversity arises from the variety of 3° amines available, ˜3000 from commercial suppliers alone. Generation of the same diversity with 4° cations using synthetic preparation^{46,47} is a bottleneck for studying self-assembly. By contrast, use of proton transfer between amine and acid allows the numbers of salts to increase both quickly and reliably, e.g., mixing 9 amines and 8 acids reliably produce 72 combinations in quantitative yields. These numbers also allow use of high throughput screening, which remains rare for supramolecular systems.⁴⁸⁻⁵² These screens have the potential for identifying building blocks that form assemblies in high yield from a broad chemical space and to help probe the underlying features controlling assembly.

Use of acid-base reactions to make ions and provide ionic stabilization to molecular recognition⁵³ and assembly⁵⁴ is not new. Early studies in anion recognition using polyaza receptors^{41,55} formed complexes with ammonium H-bonds to anions. Salt bridges^{53,56} that were studied early on,^{51,58} e.g., amidinium carboxylate,³² have now been extended to assembly.^{59,60} Contemporary uses⁶¹ involve CO₂ capture where its conversion to carbonic acid promotes proton transfer to an N-basic receptor rendering it cationic for binding HCO₃⁻ during crystallization. The inverse has been used for amine recognition. Therein, pre-incorporation of carboxylic acids into hosts facilitates proton transfer to amine guests and formation of an ionic complex.^{53,62,63} Pre-incorporation of acidic or basic sites is believed⁵³ to be a simple way to associate two ionic partners rather than trying to assemble three components and to avoid competition from ion pairing. The rarity of examples where acid, base and receptor are mixed to assemble into a single ionic species attests to this sentiment.

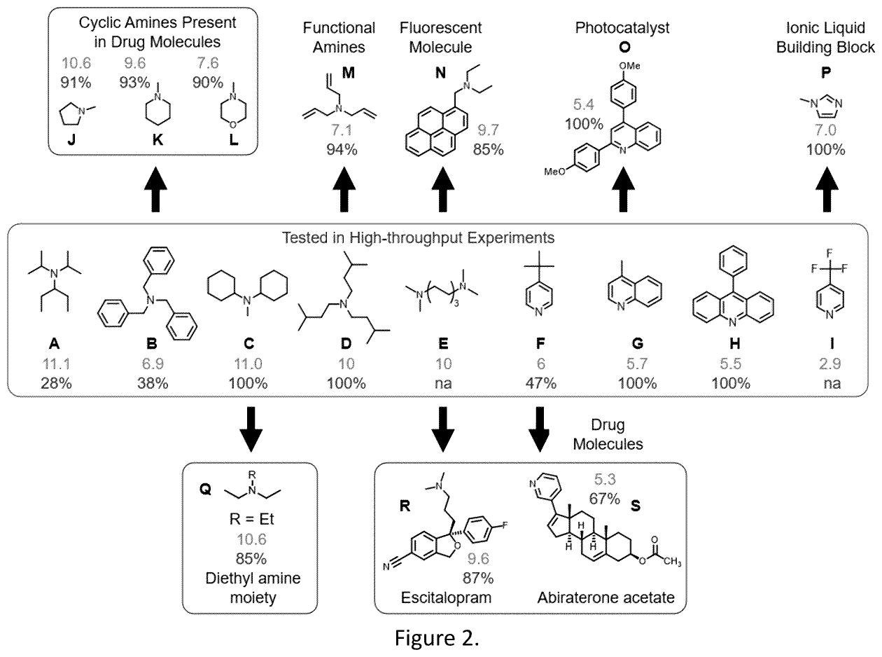

Eighty two (82) examples have been explored where three charge-neutral components (receptor, acid and amine) are mixed to undergo proton transfer and assemble into a 2:2:2 receptor-anion-cation species. Inspiration was drawn to replace the TBA⁺ cations observed in crystal structures^{64,65} formed between pi-stacked cyanostars and a dimer of phenyl phosphates (FIG. 1a) with 3° ammonium cations. It was reasoned that the exposed oxygen atoms from the phosphate dimers could serve as docking sites for the cations (FIG. 1b) but only if the 2:2 complex at the core was retained. Therein, phosphate dimers are partially stabilized by OH···O H-bonds.⁶⁵⁻⁶⁹ These are described as anti-electrostatic H-bonds⁷⁰ on account of bringing two anions together against the dictates of Coulomb's Law. A potential failure mode of assembly is therefore competition from stable, Coulomb-compliant ion pairs (FIG. 1c).^{43,44,53,71-74} This account describes both types of outcomes and how the simple preparation of diverse anions and cations allows for screening to identify privileged partners that undergo quantitative assembly. 19 N-bases (12 amines, 7 heterocycles; FIG. 2) were screened including ditopics for polymerization,⁷⁵ piperidine and analogs common in drugs⁷⁶ as well as the antidepressant CIPRALEX® (escitalopram) 77 and prostate cancer therapeutic ZYTIGA® (abiraterone acetate),⁷⁷ fluorescent compounds, quinolines used in photocatalysis,⁷⁸ and imidazoles used in ionic liquids⁷³ and as a buffer (pKₐ 6.95). Conventional 4° ammonium cations, like TBA⁺, have more reliable assembly and offer a source of diversity after clearing synthetic bottlenecks.⁴⁶ This work outlines how both anions and cations, instead of just one of them, can be tuned to enable the creation of desired assemblies by high throughput screening as well as traditional synthetic design.

### Results and Discussion

Cyanostar was mixed with phenyl phosphoric acid and dicyclohexyl-methyl amine in a stochiometric amount. The amine was selected because its protonated cation is soluble in a variety of organic solvents. The acid was selected based on confidence in its ability to form a 2:2 complex.⁶⁵ A crystal structure (FIG. 1e) shows formation of the 2:2:2 hierarchical assembly held together with three types of hydrogen bonds. At the center, is the anion dimer connected by a pair of strong and self-complementary OH···O hydrogen bonds (d_{O···O}=2.5 Å). The cyanostar dimer stabilizes the doubly charged anion dimer with 20 non-traditional CH···O hydrogen bond donors. The third layer of organization involves the charge balancing ammonium cations that are connected to the accessible oxygen atoms of the anion dimer by N⁺—H···O⁻ salt bridges (d_{N···O}=2.7 Å).

The H-bonded anion-cation contacts are classified as salt bridges.⁵⁶ These bridges involve a hydrogen bond between the specific atoms believed to serve as the seat of the formal ionic charges. In the crystal structure used for inspiration,⁶⁵ the oxygen atom in question is sp² hybridized and participates in a P═O double bond. Consistently, this bond is shorter (1.46 Å) than the P—O⁻ single bond (1.52 Å) involved in OH···O-hydrogen bonding. However, in the 2:2:2 assembly formed with the 3° cation (FIG. 1e), the phosphorous-oxygen bond lengths approach each other at 1.48 and 1.50 Å, respectively. These changes indicate that the 3° ammonium cation polarizes the phosphate, shifting charge density away from the center of the anion dimer such that it is now shared more evenly across the two oxygen atoms. The charge on the accessible oxygen acceptor atom is close to −0.5 and the association with the cation can be classified as a H-bonded salt bridge.

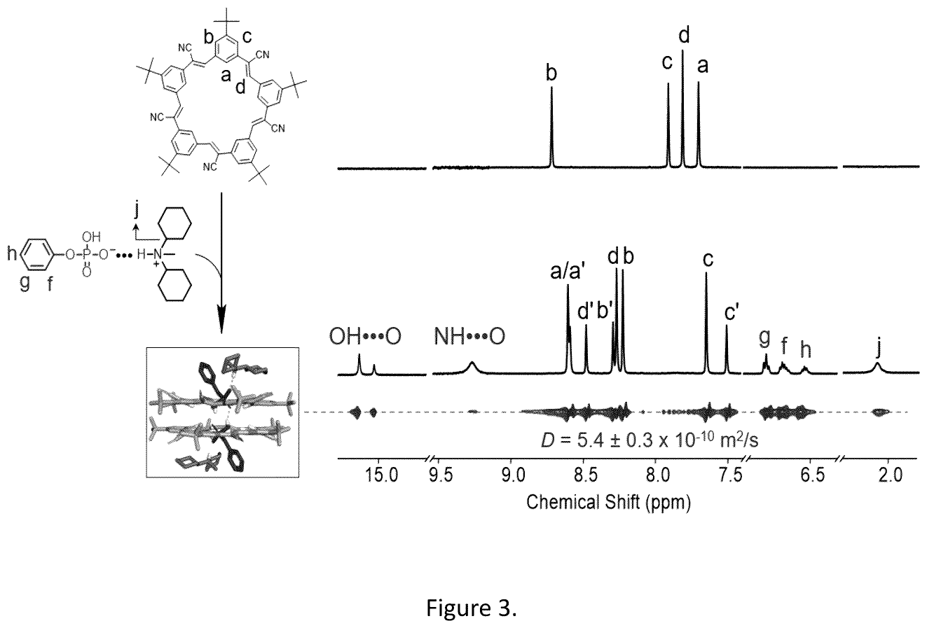

A key test for stability is whether the assembly remains intact when dissolved in solution. This stability is observed when the three components are mixed together in deuterated dichloromethane in an equimolar ratio (FIG. 3). Specifically, signatures for the CH, OH and NH H-bonds are observed in the ¹H NMR spectra (FIG. 3).

Signatures of the receptor-anion pair at the heart of the assembly are observed in the ¹H NMR spectrum. The four aromatic peaks of the cyanostar double to generate an eight-line pattern characteristic of the π-stacked cyanostar dimer as described elsewhere.⁶⁵ These eight peaks correspond to the emergence of diastereomers defined by meso (68%) and chiral (32%) combinations of pairs of cyanostars. Formation of CH H-bonds from the cyanostar to the anion are seen in the ˜0.7-ppm downfield shifts of cavity protons Ha and Hd. The protons at the center of the anti-electrostatic hydrogen bond⁶⁵ are also observed in their typical position around 15 ppm and split into diastereomeric combinations. Their 15.1-ppm position is upfield relative to the 2:2:2 assembly formed with the TBA⁺ cation (15.4 ppm)^{65,79} indicating that the OH···O H-bond is slightly weaker when assembled with the 3° cation. This interpretation is consistent with the longer O···O distance seen with the 3° ammonium (2.50 Å) compared to the 4° TBA⁺ cation (2.45 Å), and the equalization of charge across the phosphate that is induced by the cation.

Direct evidence for the NH hydrogen bond in the salt bridge is observed from the peak at 9.2 ppm. This peak was seen across many of the acid-amine combinations examined. It is often shifted upfield relative to those seen in the spectra of the ion pairs alone (12.0 ppm). The chemical shift positions of the OH, NH and CH hydrogen-bonded protons in the 2:2:2 assembly follow a rank order expected for their relative strengths (strong, medium, weak).⁴⁷

The diffusion NMR (FIG. 3) provides independent verification that the ternary assembly is formed in solution. Therein, all three components have the same diffusion coefficient (D=5.4±0.3×10⁻¹⁰ m² s⁻¹). Thus, the 2:2:2 assembly seen in the crystals is the single unitary species present in solution.

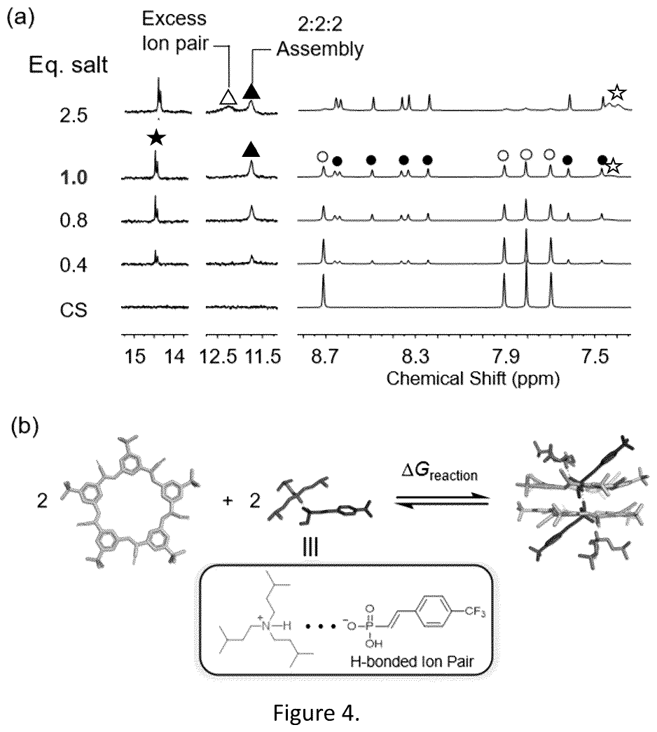

While these studies emphasize the amines, the underlying reliance on the acid-base reaction required for salt formation also adds diversity from the anion. To explore this idea, organo-phosphonic acids were examined in place of the phosphoric acid, 4-trifluoromethylphenyl vinyl phosphonic acid (CF₃-phenyl-VPA) was used.³⁰ Its corresponding phosphonate co-assembles with cyanostar as a TBA⁺ salt to form a central complex in which the oxygen atoms are exposed and available for H-bonding to 3° ammonium cations. It has a comparable acidity (pKₐ₁=2.1) to facilitate the requisite proton transfer. Despite these similarities, an equimolar mixture of the three ingredients using the triisopentylamine instead generates a mixture of product and non-assembled starting materials (FIG. 4a). Signals for the 2:2:2 species (black dots) corresponding to a 41% yield are observed. Signals for the reactants include four aromatic peaks for the free cyanostar (black open circles) and weak peaks for the ion pair stemming from the phosphonate at 7.45 ppm (red open circles). Addition of excess ion pairs (2.5 equiv.) helps drive formation of the 2:2:2 assembly. The H-bond signal at 12.25 ppm (blue open circle) for the excess ion pair is also observed. The presence of unique spectral signatures for the product and reactants provides the opportunity to better understand the equilibrium between ion pair and assembly (FIG. 4b).

For direct comparison to the exemplary phosphate assembly (FIG. 1), the phosphonic acid was combined with dicyclohexyl-methyl amine and cyanostar. It was found that several factors change the position of equilibrium including temperature, concentration, addition of TBACl, and solvent. Among these, solvent has the most dramatic impact allowing the yield to be tuned between 0 and 100%. Changing the solvent from dichloromethane to a 3:1 mixture of dichloromethane and acetonitrile, increases the proportion of 2:2:2 species from 52% to 100%, while use of tetrachloroethane decreases the yield to 0% in favor of the ion pair. Addition of small amounts of water, e.g., 0.5 μL, to the 3:1 mixture of dichloromethane and acetonitrile (540 mL) led to formation of the 2:2:2 complex with the cation dissociated in solution.

As a result of these sensitivity studies, it was found that the phosphates regularly form stable 2:2:2 assemblies in dichloromethane while the phosphonates require a more polar solvent mixture (56:44 dichloromethane-to-acetonitrile)⁸⁰ to achieve similar outcomes.

### High-Throughput Screening

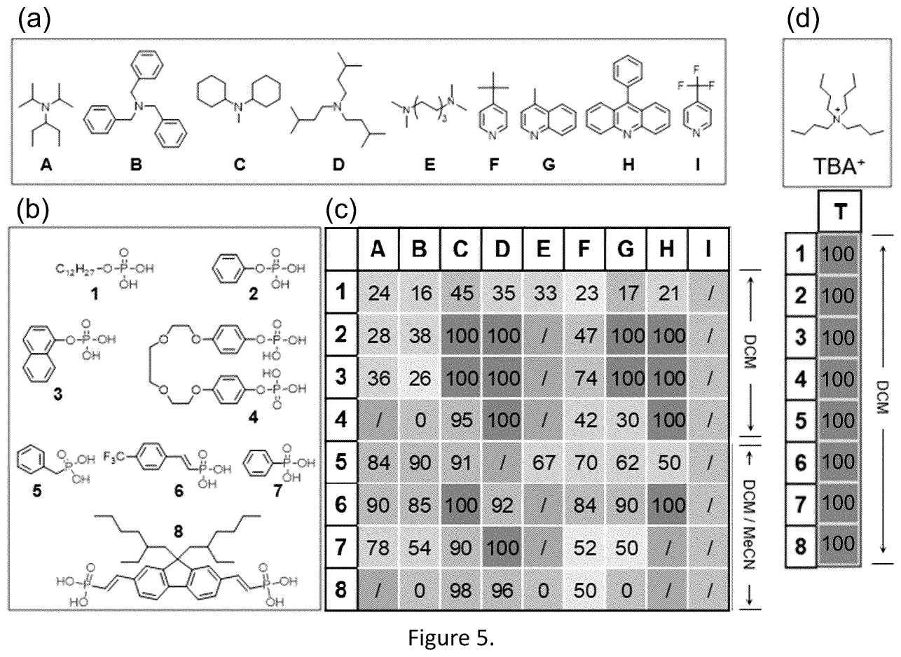

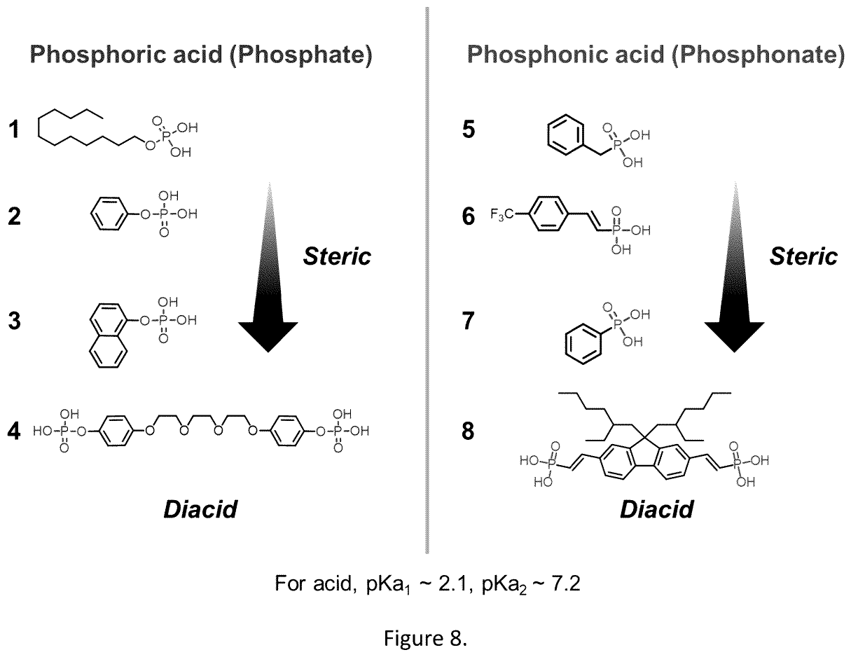

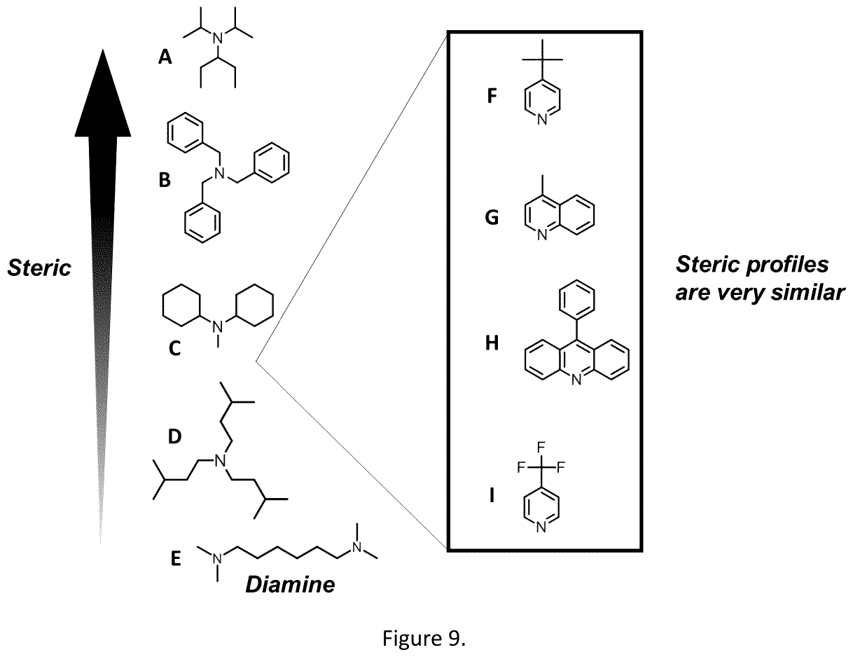

In order to access the diversity represented by the ˜3000 commercially available tertiary amines, high-throughput screening was conducted. These studies were limited to a proof-of-concept demonstration to prototype the method and evaluate its viability. 72 combinations formed by mixing 9 commercially available amines and N-bases (A-I, FIG. 5) with 8 acids of commercial or synthetic origins (1-8, FIG. 5), and combining them with cyanostar in equimolar ratios were examined. The four phosphoric acids and four phosphonic acids selected cover a range of steric profiles⁶⁵ and include two ditopic acids capable of forming supramolecular polymers⁷⁹ (FIG. 8). Pure dichloromethane is used with phosphoric acids to maximize the opportunity for identifying acid-amine partners that support high-fidelity assembly. Similarly, a 56:44 dichloromethane-acetonitrile mixture is used for the phosphonic acids. The 9 bases were selected to include diverse aliphatic amines and aromatic skeletons, a range of pKₐ values spanning 2.9 to 11.1, and different steric profiles (FIG. 9).

Across the 72 combinations, the yields of formation of the 2:2:2 assembly are assigned using NMR spectra and color coded accordingly. Of the 72 entries, 50 correspond to a heat map for the degree of formation of the 2:2:2 species. Lighter shades of grey correspond to 100% through to low degrees of assembly (16-74%). Intensities of the OH···O signal (˜15 ppm) and/or the 8-line signature of the CH···O protons in the aromatic region were used as indicators of the 2:2:2 species. This product signature is inversely correlated with the reactants, which are reflected in the four lines for the free cyanostar peaks. The NH peak for the 2:2:2 assembly has variable intensity suggesting it is not a reliable marker for either the assembly or ion pair.

The 12 entries with a slash through them are classified from observation of 100% free cyanostar peaks. The 10 grey entries show either some degree of precipitation or ambiguous proton patterns not associated with either cyanostar dimers or free cyanostar.

Thirteen (13) combinations with quantitative formation of the target ternary assembly are observed indicating a 19% success rate. Phosphates favor assembly more than the phosphonates and have a higher success rate of 28%. These 13 high-fidelity combinations represent a privileged set of receptor-anion-cation assembly partners. They include rigid cyclohexyl and flexible isopentyl substituents as well as bulky but rigid quinoline and acridine aromatics.

In this screen, preparation of the salts and assemblies (2 days) took about the same time as the collection and analysis (2 days) of the ¹H-NMR spectra. By contrast, it is estimated⁴³ that 72 salts using 4° ammonium cations would take 3-5 months to prepare. Thus, expedient acid-base preparation removes synthetic bottlenecks to reveal a bottleneck involving interpretation of the results, which is ongoing. Preliminary findings are given next.

### Interpretation of the Screening Results for Ionic Assembly

Entries composed of p-trifluoromethylpyridine (column I, pKₐ=2.92) the difference in acidity is not large enough to allow proton transfer with any of the acids (pKₐ₁˜2).⁴⁵ Success with pyridines (pKₐ 5-6) show that pKₐ differences exceeding 3 are viable for assembly.

The ditopic phosphate (row 4) displays the signature of the 2:2:2 assembly with several cations to help lay a foundation for formation of supramolecular polymers. The ditopic amine (column E) also has the potential for polymerization, however, it yields precipitation and further investigation is warranted.

The OH···O H-bond signature from a 3:2 cyanostar: anion assembly present as a minor species is also observed when combining alkyl-phosphoric acid 1 with amines C and D. This observation is consistent with a previous account⁶⁵ that the small size of the alkyl substituent on the phosphate allows generation of a triple stack of macrocycles. The extra macrocycle stabilizes the anion dimer and introduces steric bulk that will disfavor further assembly with cations. Despite this situation, this and other species may be alternative targets of optimization using acid-base screening.

Some combinations indicate that the amine's sterics play a role in controlling the yield of assembly. For instance, tribenzyl amine (B) and dicyclohexyl-methyl amine (C) display different degrees of assembly in the presence of phosphonic acid 6. When combined in dichloromethane 0% and 75% yields of the corresponding 2:2:2 assemblies are observed. To investigate the steric origin of these differences, molecular models of the 2:2:2 complexes were generated. For this purpose, molecular mechanics was used as an expedient way to evaluate the idea. Briefly, the 2:2 complex formed between anion 6⁻ and cyanostar was used and then the geometry was frozen in the one obtained from the crystal structure.³⁰ The two cations, BH⁺ and CH⁺, were docked to the 2:2 complex and the NH···O bond distance (2.7 Å) was constrained to the one observed in the crystal of the 2:2:2 assembly (FIG. 1e). In both cases, the three substituents on the cation need to be rotated away from the NH donor to expose it for H-bonding and to minimize steric contacts. The conformation of this H-bonded cation was optimized subject to these constraints, excised from the assembly and its energy calculated. The geometry of the ion pair was also optimized but without constraints. The cation was also excised and the energy of its conformation calculated. The strain energy associated with changing the geometry of the cation in the ion pair (reactant) to the one in the 2:2:2 assembly (product) was evaluated. Consistent with a lower degree of assembly, it was observed that a greater strain energy (102 kJ mol⁻¹) is necessary with cation BH⁺ compared to cation CH⁺ (55 kJ mol⁻¹). Similar effects were noted by Wilson^{81,82} looking at the stabilities of ion pairs between ammonium and various anions.

These experiments also revealed cross-dependencies such that 100% assembly depends not only on the structure of the cation, but on its combination with specific anions. Combinations C6 and D7 have 100% yields suggesting C7 and D6 would also favor assembly. However, their yields are 50% or less. Cooperativity between assembly partners must also be at play.

For the fluorescent acridinium cation (H) formed after proton transfer, different fluorescence outcomes are observed when changing from acid 1 to acid 4 when examined under UV. These observations indicate the potential for environmentally responsive behaviors in the future.

Triethylamine (FIG. 2, TEA) was also examined on account of the simplicity with which the diethylamino moiety [—NEt₂] can be introduced into various building blocks by alkylation.⁸³⁻⁸⁹ TEA also supports high fidelity formation of the ternary assembly when combined with phenyl phosphate and cyanostar. Inspired by this example, a diethylamino-functionalized pyrene (N) was synthesized using N-alkylation. This amine was observed to co-assemble with phenyl phosphoric acid and cyanostar to afford the ternary assembly (85%). This promising result opens a pathway to deliver various functionalities to these 2:2:2 assemblies by synthetic design.

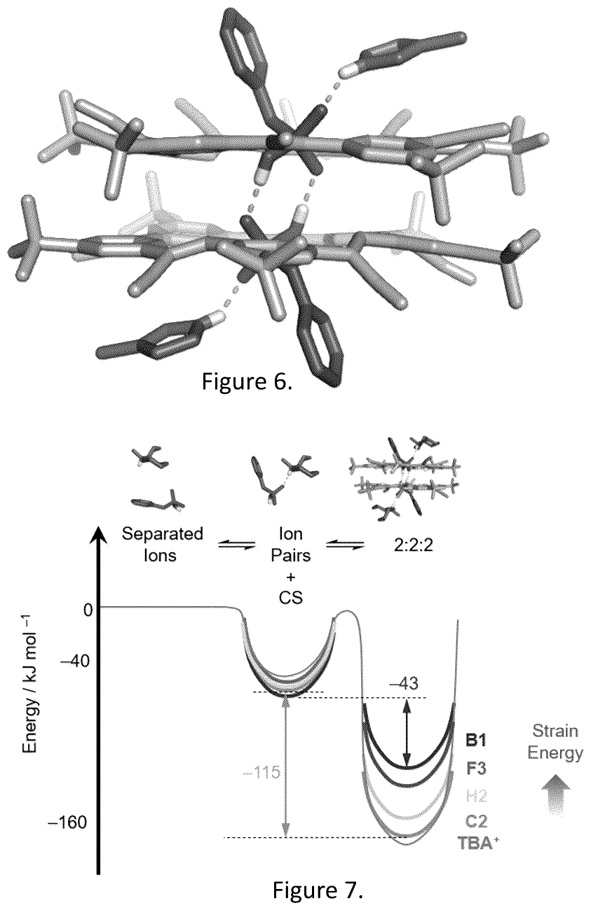

Observations obtained from high throughput experiments as well as the molecular skeletons were taken advantage of to expand the diversity of amines (FIG. 2) for ternary assembly. To simplify assessments, phenyl phosphoric acid was used in all cases. The scope of the amines was expanded to cyclic ones that are ubiquitous in pharmaceutical chemistry⁷⁶ including 1-methylpyrrolidine (J), 1-methylpiperidine (K) and 4-methylmorpholine (L) with assembly yields of 91, 93, 90%, respectively. Triallylamine M, bearing free alkene groups ready for further modification using olefin metathesis undergoes assembly (94%). A reported quinoline photocatalyst⁷⁸ O was used and it was found that it forms a high-fidelity ternary assembly (100%). It was observed that imidazole, with wide use across various areas of ionic liquids, N-heterocyclic carbene synthesis, and as pH buffers, also assembles with 100% yield. The 2:2:2 crystal structure shows the imidazolium p stacking with the cyanostar at a distance of ˜3.75 Å and titled 21° from the macrocycle's mean plane (FIG. 6). Finally, drug molecules escitalopram (R) and abiraterone acetate(S) bearing trimethylamine or pyridine moieties, respectively, supports the ternary assembly with 87 and 67% yields. The lower yield from S likely originates from its large steroidal structure. Thus, the diversity of options has been explored to include 10 unique compound classes offering broad access to areas spanning chemistry, materials and biology. This demonstration of diversity shows the promising future of this simple assembly methodology.

Studies with 1° and 2° amines show that they do not support formation of 2:2:2 species (data not shown). For these tests, phenyl phosphoric acid (2) and the 4-trifluoromethylphenyl vinyl phosphonic acid (6) were selected for combination with 1° and 2° amines, respectively. After acid-base reaction, the salts have poor solubility in dichloromethane as noted by Shinkai.³¹ Amines with longer alkyl chains, e.g., changing the alkyl from dibutylamine to didodecylamine, show modest improvements. These observations are attributed to stronger ion pairing and multiple NH hydrogen bonds.^{43,72} The highest percentage of assembly is ˜20% when the soluble salt of phenyl phosphate and didodecyl ammonium is combined with cyanostar. These observations suggest that two or more H-bonds in the ion pair are sufficient to outcompete formation of the assembly.

Comparison and Scope for Assembly with Quaternary (4°) Ammonium Cations

Motivated by these studies, the aprotic 4° TBA⁺ cation was re-examined and compared to the 3° cations formed by acid-base chemistry. Previous work with organophosphates⁶⁵ had found the TBA⁺ to be dissociated from the 2:2 assemblies but it was recognized that use of a more polar medium (2:1 CD₂Cl₂:CD₃CN) likely promoted that outcome.⁶⁵ The DOSY data of the assembly formed upon mixing cyanostar with phenyl phosphate as the TBA⁺ salt in dichloromethane show each component has the same diffusion coefficient. Thus, the 2:2:2 species is also formed with the 4° TBA⁺ cation in dichloromethane.

The TBA⁺ cation is routinely used to disfavor ion pairing and promote anion complexation. Thus, its 2:2:2 assembly is expected to be more stable relative to the ion pairs than what is possible with 3° ammonium cations. Using a competition study with a 1:1 mixture of dicyclohexylmethyl ammonium and TBA⁺ as phenyl phosphate salts, ¹H-NMR peak shifts that slightly favor assembly with the 4° TBA⁺ cation are observed. The resulting 60:40 ratio indicates a small energy difference of 1 kJ mol⁻¹.

These findings promote consideration of 4° ammonium and other aprotic cations as additional sources of chemical diversity. Exploration of small-molecule, ionic isolation lattices⁹ and the charge-by-charge assemblies^{7,10} of Maeda using anion salts of cationic dyes also suggest the scope for ionic assembly is broader than considered with the 3° ammonium cations alone. To this end, a new column was added to the screen (FIG. 5d) to highlight this possibility.

One key difference between 3° and 4° ammonium cations is their synthetic preparation. Use of 3° ammonium cations allows acid-base chemistry to add new combinations reliably and without fuss. The salts produced by acid-base chemistry do not require any special synthetic methods and rely on simple mixing and vacuum drying. While they may be recrystallized to remove any residual acid or amine and raise purities, they are not necessarily any different than most commercial supplies that offer >98% purity. By contrast, the synthesis of 4° cations⁴³ requires N-alkylation of 3° amines and metathesis to access diverse combinations. The time investments needed for identifying reaction and purification conditions inhibit screening and investigations of how the cation's structure impacts the final assembly. Yet these investments need not be prohibitive. Rather, the development of diverse 4° ammonium cations, possibly leveraging efforts in catalyst design⁹⁰ and the elaboration of other aprotic N⁺-based cations, is expected to be a fruitful area of exploration.

### Energetics of Assembly

According to the different yields of 2:2:2 assembly when using the 3° and 4° ammonium cations, the form of an energy profile of the system can be estimated (FIG. 7). The full energy landscape has been described with HSO₄⁻ forming 2:2:2 species.⁶⁴ In the present case, however, the following estimates and assumptions are used. First, the ion pairing between TBA⁺ and phenyl phosphate was measured at −44 kJ mol⁻¹ to help provide one anchor point for the energy profile for this cation (grey line, FIG. 7). Second, ion pairing is believed to be stronger for the 3° ammonium cations³¹ and to depend on the identity⁸² of anion and cation leading to the energy spread shown in the blue wells for the salts of B1, C2, F3, and H2. Third, the 2:2:2 assembly using dicyclohexylmethyl ammonium with phenyl phosphate (C2) is only slightly weaker (1 kJ mol⁻¹) than with TBA⁺. Fourth, the 0-100% populations are used to generate reaction free energies ΔGᵣₓₙ. These span from less than 0 kJ mol⁻¹ to more than −115 kJ mol⁻¹.⁹¹

A large range in the reaction free energies of assembly is observed. It is believed that the variations in the energies of the products are bigger than in the reactants. One source of the difference between the reactants (ion pair and cyanostar) and product (2:2:2) is the additional strain energy in the cation needed for formation of the assembly (vide supra). The calculated strain energies on bulky BH⁺ and compact CH⁺ were used to situate the bottom of the energy wells of their 2:2:2 assemblies ˜40 kJ mol⁻¹ apart from each other. In addition, a competition study suggests the 2:2:2 with aliphatic ammonium C2 is more stable than aromatic H2 by 3 kJ mol⁻¹. It is believed that the strain energy is the biggest source of variation in both reaction free energies and overall stabilities of the 2:2:2 assemblies. It follows, therefore, that stability depends on the identity of the 3° cation. This interpretation and the observation the identity of both anion and cation cooperatively impact stability are difficult to investigate due to current synthesis bottlenecks.

### Conclusion

Simple acid-base chemistry and stable anion-receptor complexes were used to provide access to chemically diverse ammonium cations for hierarchical ionic assembly. The 3° ammonium cations are observed to dock with the primary 2:2 cyanostar-anion assembly where the cations serve as a secondary layer of structural ordering in the hierarchical assembly. The ternary 2:2:2 assembly is in a competitive equilibrium with the simple 1:1 ion pair plus free cyanostar. This equilibrium depends on the stability of the anion-receptor complex at the core. Phosphates show a greater number of high-yield assemblies relative to phosphonates. The position of equilibrium is highly sensitive to the solvent used, with changes in polarity capable of turning assembly both on and off by favoring either the 2:2:2 assembly or ion pair. A high-throughput screen of 72 combinations was used to evaluate a diverse set of acids and bases and their corresponding ionized forms as anions and cations. The screening was used to rapidly identify 13 partners that produce the assembly in quantitative yields. The diethylamine moiety, —NEt₂, was identified as a functional group that can be conveniently introduced using high-yielding covalent chemistry, e.g., N-alkylation. Diversity was extended to cationic forms of photocatalysts, drug molecules, fluorophores and a component of ionic liquids to demonstrate straightforward integration with other sub-fields of chemistry. The simplicity of preparation allows a manual NMR screening process to be conducted. The NMR output affords insight into the structural state of assembly and aids understanding of molecule-level details, e.g., the effect of sterics and strain on yield. These studies also identified 4° ammonium cations as potential sources of diversity to explore in the future after removing synthesis bottlenecks. The systems presented is exemplary of the benefits accruing when diverse cations and anions are combined into a simple assembly pipeline to produce hierarchical molecules and materials. Future applications include use in fluorescent materials and supramolecular polymers.

### Methods

Reagents were obtained from commercial suppliers and used as received unless otherwise indicated. Two vinyl phosphonic acids were synthesized according to literature. Column chromatography was performed on silica gel (160-200 mesh, Sorbent Technologies, USA). Thin-layer chromatography (TLC) was performed on pre-coated silica gel plates (0.25 mm thick) and observed under UV light. 1D Nuclear magnetic resonance (NMR) spectra and titrations were recorded on Varian Inova (500 and 600 MHz) and Bruker Avance Neospectrometers (500 MHz) at room temperature (298 K). Chemical shifts were referenced to residual solvent peaks.

### Experimental Method for NMR Titration

In a forward titration, a solution of cyanostar macrocycle was prepared in an NMR tube sealed with a silicone septum and an initial spectrum was taken. A solution of phosphate or phosphonate ammonium salt was also prepared and added to the solution of cyanostar macrocycle with known quantities, the spectrum was recorded after each addition. In a reverse titration, a solution of ammonium salt was prepared in an NMR tube and a solution of cyanostar macrocycle was added to the solution of ammonium salt. All the spectra data were analyzed by using MestReNova software.

### Experimental Method for Diffusion NMR

The diffusion coefficients were then obtained based on the method of pulse gradient spin echo (PGSE) experiments. Aromatic regions were analyzed in this way to determine diffusion coefficients using Vnmrj's analysis. Average diffusion coefficients and errors were generated from multiple peaks used in analyses.

### Standard Procedure of Converting Phosphonic or Phosphoric Acid to its Ammonium Salt.

Using vinyl phosphonate ammonium salt preparation as an example. The ammonium phosphonate salt was formed by titrating the corresponding acid methanol solution with aliquots of tertiary amine dichloromethane solution until deprotonation was complete as verified by ¹H NMR spectroscopy based on the integration of amine's proton and proton of vinyl group from phosphonic acid. Then the solvent was evaporated, and the resulting salt was dried under vacuum for 1 day at room temperature before using for complexation.

Regarding the purity of the prepared ammonium salts. The procedure involves mixing acids and amines. Without recrystallization, small (trace) amounts of excess species (acids or amines) should exist, even though the corresponding NMR signals are not observed. Most of the salts are made in this way. In two cases (C2, C6), the impact of salt preparation on the outcome of assembly was compared. Using NMR titration, the acid-base reaction was monitored using NMR signals of acid and amine to ensure the stoichiometric mixing of acid and amine on 10-mg scale. Identical yields of assembly were obtained using either mixing or titration.

### Procedure of High-Throughput Experiments

6 mM acid solutions in methanol and amine solutions in dichloromethane were prepared. To prepare the salt, stoichiometric amount of acid and amine solutions were mixed in NMR tube and dried on vacuum overnight. The dry phosphate ammonium then was dissolved in deuterium dichloromethane (2.6 mM), and the phosphonate ammonium was dissolved in 35:65 deuterium dichloromethane: deuterium acetonitrile (2.6 mM). ¹H NMR spectra were collected. Then stoichiometric amount of 6 mM cyanostar in deuterium dichloromethane was added to the salt solution to make a 1.8 mM solution (total volume is 650 μL) and ¹H NMR spectra were collected. In the case of phosphonate, after addition of cyanostar, the solvent ratio is 56:44 dichloromethane:acetonitrile.

Studies of Factors that Affect the Equilibrium Between Ion Pair and 2:2:2 Assembly

A number of factors change the position of equilibrium. These factors were tested using the combination of CF₃-phenyl-VPA, dicyclohexylmethylamine and cyanostar.

Decreasing the temperature from 50 to −50° C. enhances the proportion of the assembly from 25% to 63%. Presumably, lower temperature favors the enthalpic product with more components.

Concentration also has an impact. At very low concentration, i.e., 0.1 mM, only 10% of assembly presents. When the concentration increases to 0.5 mM, 62% of assembly was observed. However, increasing the concentration from 0.5 to 10 mM shows a modest impact by lowering the proportion of the assembly from 62 to 39%.

Solvent has a dramatic impact. Among the series of solvents tested, the proportion of assembly can be varied between 0 and 100%. For example, changing from dichloromethane to chloroform, the proportion of 2:2:2 species drop from 52% to 44%. Presumably, the lower polarity solvent favors the side of the equilibrium electrostatics plays a more dominant role on stabilization. In this case, ion pairing over H-bonding. Consistently, increasing polarity by adding up to 25% by volume of acetonitrile to dichloromethane shifts the equilibrium to favor formation of only the 2:2:2 assembly. Interestingly, in this mixing experiment, if the acetonitrile is the normal deuterium one, which contains trace amount of water, 1:3 mixture of acetonitrile and dichloromethane results in quantitative formation of the 2:2 species. [0.2 microliters of water in a 1:3 mixture of acetonitrile and dichloromethane (540 microliters) based on NMR integration] However, when a dried acetonitrile is used, 1:3 mixture of acetonitrile and dichloromethane gives pure 2:2:2 assembly, and a smooth transition from the 2:2:2 to the 2:2 species can be observed by the addition of 0.5 microliters of water. Addition of water to dichloromethane, while immiscible, dissociates most of the high-fidelity assembly formed between phenylphosphate, dicyclohexylmethyl ammonium and cyanostar, into the ion pair and free cyanostar with 15% residual ternary assembly.

Addition of the TBA⁺ cation also generates signature seen with only this salt alone. This cation was added as the chloride (Cl⁻) salt, which is too weak to compete with cyanostar binding. It is believed that a more stable ion pair forms between 3° dicyclohexylmethylammonium cation and chloride by in situ metathesis. Consistently, the NH signal shifts to the similar position (11.7 ppm) of the one observed when a neat sample of dicyclohexylmethylammonium chloride is made in dichloromethane (11.6 ppm).

Some combinations suggest a steric effect of amine, such as tribenzylamine (B) and dicyclohexylmethylamine (C) that display different degrees of assembly in the presence of trifluoromethylphenyl vinyl phosphonic acid (6). When combined together in dichloromethane 0% and 75% assembly are observed. To investigate these differences, molecular models of the 2:2:2 complexes were made by replacing the cation C with the ones formed from B and subjecting it to a geometry optimization. To generate the structures, the crystal structure of the 2:2 complex involving anion 6 and the 4° TBA⁺ cation was used as a starting point. The TBA⁺ cations were removed. To this core was docked the 3° cations of interest. The initial geometry of the cation was based on the lowest energy conformer of the cation alone. The substituents were rotated manually to avoid steric clashes. In both cases, the substituents are sufficiently bulky that there was only one major option for rotating them out of the way and to leave the NH bond exposed for H-bonding.

The geometry of this 2:2:2 assembly was optimized under the following constraints. The 2:2 core was frozen, and the NH···O distance was fixed at 2.7 Å to match the one observed in the crystal structure (FIG. 1). The geometry was optimized using molecular mechanics. To test the validity and accuracy of this method, the dependence of the conformation was explored by relaxing certain constraints. When the H-bond distance was allowed to relax, it changed from 2.7 to 2.6 Å. The overall conformation did not significantly change with the relaxation of this constraint, but the energy of the cation conformer from the assembly before and after relaxation changed from 229 to 205 kJ mol⁻¹.

The cation was then excised, and the energy of its conformation determined using molecular mechanics. For the energy of the conformer present in the ion pair, the two ions were prepared as linear ion pairs and allowed to minimize their energy using molecular mechanics. Again, the cation was excised, and its energy evaluated. The conformations of cations B and C that are generated when they form 2:2:2 assemblies were compared to those of the ion pair. The modelling shows the strain energy needed to form the conformation of cation B in the assembly (102 kJ mol⁻¹) is much higher in energy than it is for cation C (55 kJ mol⁻¹). This finding correlates with the degree of assembly of each cation.

### X-Ray Crystallography

Tetrabutylammoium phenylphosphate and cyanostar (label 21193)

### Data Collection

The data collection was carried out using Mo K radiation (=0.71073 Å, ImS micro-source) with a frame time of 10 seconds and a detector distance of 40 mm. A collection strategy was calculated and complete data to a resolution of 0.82 Å with a redundancy of 4.6 were collected. The frames were integrated with the Bruker SAINT[S4] software package using a narrow-frame algorithm. The integration of the data using a triclinic unit cell yielded a total of 185488 reflections to a maximum θ angle of 25.17° (0.84 Å resolution), of which 15500 were independent (average redundancy 11.967, completeness=99.4%, Rint=16.11%, Rsig=6.42%) and 7062 (45.56%) were greater than 2σ(F2). The final cell constants of a=15.6034(10) Å, b=16.7014(10) Å, c=17.4991(10) Å, α=101.929(2) °, β=102.556(2)°, γ=90.688(2) °, volume=4347.3(5) Å³, are based upon the refinement of the XYZ-centroids of 9682 reflections above 20σ(I) with 4.347°<2θ<37.82°. Data were corrected for absorption effects using the Multi-Scan method (SADABS).[S5] The ratio of minimum to maximum apparent transmission was 0.904. The calculated minimum and maximum transmission coefficients (based on crystal size) are 0.6735 and 0.7452. Please refer to Table 1 for additional crystal and refinement information.

### Structure Solution and Refinement

The space group P-1 was determined based on intensity statistics and systematic absences. The structure was solved using the SHELX suite of programs and refined using full-matrix least-squares on F² within the OLEX2 suite. An intrinsic phasing solution was calculated, which provided most non-hydrogen atoms from the E-map. Full-matrix least squares/difference Fourier cycles were performed, which located the remaining non-hydrogen atoms. All non-hydrogen atoms were refined with anisotropic displacement parameters. The hydrogen atoms were placed in ideal positions and refined as riding atoms with relative isotropic displacement parameters. The final full matrix least squares refinement converged to R1=0.1335 and wR2=0.4271 (F², all data). The goodness-of-fit was 1.347. On the basis of the final model, the calculated density was 1.077 g/cm³ and F(000), 1515 e⁻.
Dicyclohexylmethyl amine, phenylphosphoric acid and cyanostar (label 23060)

### Data Collection

A colorless crystal (approximate dimensions 0.332×0.164×0.086 mm³) was placed onto the tip of a MiTeGen loop and mounted on a Bruker Venture D8 diffractometer equipped with a PhotonIII detector at 173(2) K.

The data collection was carried out using Mo K radiation (graphite monochromator) with a frame time of 1, 30, 45 and 60 seconds and a detector distance of 4.00 cm. A collection strategy was calculated and complete data to a resolution of 0.80 Å with a redundancy of at least 7 were collected. Fourteen major sections of frames were collected with 1° and scans. A total of 2062 frames were collected. The structure was twinned with a domain ratio of 96:4, twin law by the rows −1 0 0 −0.208 1 −0.906, 0 0 −1, 180° about reciprocal axis 0 1 0. The frames were twin-integrated with the Bruker SAINT software package using a narrow-frame algorithm. The integration of the data using a triclinic unit cell yielded a total of 242286 reflections to a maximum θ angle of 26.42° (0.80 Å resolution), of which 16674 were independent (average redundancy 14.531, completeness=99.7%, Rᵢₙₜ=15.36%, R_{sig}=4.25%) and 12523 (75.10%) were greater than 2σ(F²). The final cell constants of a=13.4009(6) Å, b=18.3698(8) Å, c=19.2446(9) Å, α=63.9072(15)°, β=73.6262(15)°, γ=78.9974(15) °, volume=4069.8(3) Å³, are based upon the refinement of the XYZ-centroids of 9895 reflections above 20σ(I) with 4.404°<2θ<52.69°. Data were corrected for absorption effects using the Multi-Scan method (TWINABS). The ratio of minimum to maximum apparent transmission was 0.760. The calculated minimum and maximum transmission coefficients (based on crystal size) are 0.9510 and 0.9870. Please refer to Table 1 for additional crystal and refinement information.

### Structure Solution and Refinement

The space group P-1 was determined based on intensity statistics and the lack of systematic absences. The structure was solved and refined using the SHELX suite of programs. An intrinsic-methods solution was calculated, which provided most non-hydrogen atoms from the E-map. Full-matrix least squares/difference Fourier cycles were performed, which located the remaining non-hydrogen atoms. All non-hydrogen atoms were refined with anisotropic displacement parameters. The hydrogen atoms were placed in ideal positions and refined as riding atoms with relative isotropic displacement parameters. The final anisotropic full-matrix least-squares refinement on F² with 1202 variables converged at R1=9.38%, for the observed data and wR2=22.58% for all data; only the major data for the major component of the twin were used. The goodness-of-fit was 1.050. The largest peak in the final difference electron density synthesis was 0.906 e−/Å³ and the largest hole was −0.921 e−/Å³ with an RMS deviation of 0.066 e−/Å³. On the basis of the final model, the calculated density was 1.179 g/cm³ and F(000), 1548 e−. Hydrogen bonding was observed. Details are in the tables.

TABLE 1
Crystal data and structure refinement

| | |
|---|---|
| Empirical formula | C88.50 H110 Cl1.50 N6 O4.25 P |
| Formula weight | 1409.96 |
| Crystal color, shape, size | colourless block, 0.15 × 0.12 × 0.11 mm³ |
| Temperature | 220.0 K |
| Wavelength | 0.71073 Å |
| Crystal system, space group | Triclinic, P-1 |
| Unit cell dimensions | a = 15.6034(10) Å α = 101.929(2)°. |
| | b = 16.7014(10) Å β = 102.556(2)°. |
| | c = 17.4991(10) Å γ = 90.688(2)°. |
| Volume | 4347.3(5) Å³ |
| Z | 2 |
| Density (calculated) | 1.077 g/cm³ |
| Absorption coefficient | 0.127 mm⁻¹ |
| F(000) | 1515 |
| Data collection | |
| Diffractometer | Bruker VENTURE D8 |
| Theta range for data collection | 1.911 to 25.168°. |
| Index ranges | −18 <= h <= 18, −19 <= k <= |
| | 19, −20 <= 1 <= 20 |
| Reflections collected | 185488 |
| Independent reflections | 15500 [Rint = 0.1611] |
| Observed Reflections | 7062 |
| Completeness to theta = | 99.4% |
| 25.168° | |
| Solution and Refinement | |
| Absorption correction | Semi-empirical from equivalents |
| Max. and min. transmission | 0.7452 and 0.6735 |
| Solution | Intrinsic methods |
| Refinement method | Full-matrix least-squares on F² |
| Weighting scheme | w = [σ²Fσ² + AP² + BP]⁻¹, with |
| | P = (Fo² + 2 Fc²)/3, A = 0.200, B = 0.000 |
| Data/restraints/parameters | 15500/2177/1205 |
| Goodness-of-fit on F² | 1.347 |
| Final R indices [I > 2σ(I)] | R1 = 0.1335, wR2 = 0.3619 |
| R indices (all data) | R1 = 0.2231, wR2 = 0.4271 |
| Largest diff. peak and hole | 1.107 and −1.081 e.Å⁻³ |

### REFERENCES FOR EXAMPLE 1

1. Kim, S. K., and Sessler, J. L. (2010). Ion pair receptors. Chem. Soc. Rev. 39, 3784. 10.1039/c002694h.
2. He, Q., Vargas-Zuniga, G. I., Kim, S. H., Kim, S. K., and Sessler, J. L. (2019). Macrocycles as Ion Pair Receptors. Chem. Rev. 119, 9753. 10.1021/acs.chemrev.8b00734.
3. Liang, L., Zhao, W., Yang, X. J., and Wu, B. (2022). Anion-Coordination-Driven Assembly. Acc. Chem. Res. 55, 3218. 10.1021/acs.accounts.2c00435.
4. Zhang, L., Li, B., Li, R., Wang, Y., Ye, S., Zhang, P., and Wu, B. (2023). Spontaneous Resolution of Chiral Janus-Type Double-Layered Metallocyclic Strips Incorporating Mobius Ring and Circular Helicate. J. Am. Chem. Soc. 145, 18221. 10.1021/jacs.3c05746.
5. Bai, X., Jia, C., Zhao, Y., Yang, D., Wang, S. C., Li, A., Chan, Y. T., Wang, Y. Y., Yang, X. J., and Wu, B. (2018). Peripheral Templation-Modulated Interconversion between an A(4) L(6) Tetrahedral Anion Cage and A(2) L(3) Triple Helicate with Guest Capture/Release. Angew. Chem., Int. Ed. 57, 1851. 10.1002/anie.201712080.
6. Li, B., Zhang, W., Lu, S., Zheng, B., Zhang, D., Li, A., Li, X., Yang, X. J., and Wu, B. (2020). Multiple Transformations among Anion-based A(2n) L(3n) Assemblies: Bicapped Trigonal Antiprism A(8) L(12), Tetrahedron A(4) L(6), and Triple Helicate A(2) L(3) (A=Anion). J. Am. Chem. Soc. 142, 21160. 10.1021/jacs.0c10346.
7. Haketa, Y., Sasaki, S., Ohta, N., Masunaga, H., Ogawa, H., Mizuno, N., Araoka, F., Takezoe, H., and Maeda, H. (2010). Oriented salts: dimension-controlled charge-by-charge assemblies from planar receptor-anion complexes. Angew. Chem., Int. Ed. 49, 10079. 10.1002/anie.201006356.
8 Haketa, Y., Bando, Y., Takaishi, K., Uchiyama, M., Muranaka, A., Naito, M., Shibaguchi, H., Kawai, T., and Maeda, H. (2012). Asymmetric induction in the preparation of helical receptor-anion complexes: ion-pair formation with chiral cations. Angew. Chem., Int. Ed. 51, 7967. 10.1002/anie.201202196.
9. Benson, C. R., Kacenauskaite, L., VanDenburgh, K. L., Zhao, W., Qiao, B., Sadhukhan, T., Pink, M., Chen, J., Borgi, S., Chen, C.-H., et al. (2020). Plug-and-Play Optical Materials from Fluorescent Dyes and Macrocycles. Chem 6, 1978. 10.1016/j.chempr.2020.06.029.
10. Haketa, Y., Komatsu, K., Sei, H., Imoba, H., Ota, W., Sato, T., Murakami, Y., Tanaka, H., Yasuda, N., Tohnai, N., and Maeda, H. (2024). Enhanced solid-state phosphorescence of organoplatinum pi-systems by ion-pairing assembly. Chem. Sci. 15, 964. 10.1039/d3sc04564a.
11. Skala, L. P., Aguilar-Enriquez, X., Stern, C. L., and Dichtel, W. R. (2023). Dissipative crystallization of ion-pair receptors. Chem 9, 709. 10.1016/j.chempr.2022.12.010.
12. Zhang, W., Jin, W., Fukushima, T., Saeki, A., Seki, S., and Aida, T. (2011). Supramolecular Linear Heterojunction Composed of Graphite-Like Semiconducting Nanotubular Segments. Science 334, 340. doi: 10.1126/science.1210369.
13. Aida, T., Meijer, E. W., and Stupp, S. I. (2012). Functional Supramolecular Polymers. Science 335, 813. 10.1126/science.1205962.
14. Yu, Z., Tantakitti, F., Yu, T., Palmer, L. C., Schatz, G. C., and Stupp, S. I. (2016). Simultaneous covalent and noncovalent hybrid polymerizations. Science 351, 497. 10.1126/science.aad4091.
15. Kleine-Kleffmann, L., Stepanenko, V., Shoyama, K., Wehner, M., and Wurthner, F. (2023). Controlling the Supramolecular Polymerization of Squaraine Dyes by a Molecular Chaperone Analogue. J. Am. Chem. Soc. 145, 9144. 10.1021/jacs.3c01002.
16. Wang, S., Huang, Z., Li, A., Zhao, Y., Zuo, W., Li, Y., Miao, H., Ma, J., Sun, W., Wang, X., et al. (2021). Crown Ether Functionalized Potassium-Responsive Anionocages for Cascaded Guest Delivery. Angew. Chem., Int. Ed. 60, 9573. 10.1002/anie.202100441.
17. Barbera, L., De Plano, L. M., Franco, D., Gattuso, G., Guglielmino, S. P. P., Lando, G., Notti, A., Parisi, M. F., and Pisagatti, I. (2018). Antiadhesive and antibacterial properties of pillar[5]arene-based multilayers. Chem. Commun. 54, 10203. 10.1039/c8cc05659e.
18. Hutskalov, I., Linden, A., and Coric, I. (2023). Directional Ionic Bonds. J. Am. Chem. Soc. 145, 8291. 10.1021/jacs.3c01030.
19. Skala, L. P., Stern, C. L., Bancroft, L., Moisanu, C. M., Pelkowski, C., Aguilar-Enriquez, X., Swartz, J. L., Wasielewski, M. R., and Dichtel, W. R. (2023). A modular platform for the precise assembly of molecular frameworks composed of ion pairs. Chem 9, 1208. 10.1016/j.chempr.2023.01.011.
20. Moisanu, C. M., Jacobberger, R. M., Skala, L. P., Stern, C. L., Wasielewski, M. R., and Dichtel, W. R. (2023). Crystalline Arrays of Copper Porphyrin Qubits Based on Ion-Paired Frameworks. J. Am. Chem. Soc. 145, 18447. 10.1021/jacs.3c04786.
21. Yang, P., Wang, J., Jia, C., Yang, X. J., and Wu, B. (2013). Dinuclear Chloride-Binding Foldamers Based on Fluorescent Oligoureas. Eur. J. Org. Chem. 2013, 3446. 10.1002/ejoc.201300043.
22. Silver, E. S., Rambo, B. M., Bielawski, C. W., and Sessler, J. L. (2014). Reversible anion-induced cross-linking of well-defined calix[4]pyrrole-containing copolymers. J. Am. Chem. Soc. 136, 2252. 10.1021/ja4123895.
23. Caballero, A., Swan, L., Zapata, F., and Beer, P. D. (2014). Iodide-induced shuttling of a halogen- and hydrogen-bonding two-station rotaxane. Angew. Chem., Int. Ed. 53, 11854. 10.1002/anie.201407580.
24. Gavette, J. V., Evoniuk, C. J., Zakharov, L. N., Carnes, M. E., Haley, M. M., and Johnson, D. W. (2014). Exploring Anion-Induced Conformational Flexibility and Molecular Switching in a Series of Heteroaryl-Urea Receptors. Chem. Sci. 5, 2899. 10.1039/C₄SC00950A.
25. Wang, H., Fang, S., Wu, G., Lei, Y., Chen, Q., Wang, H., Wu, Y., Lin, C., Hong, X., Kim, S. K., et al. (2020). Constraining Homo- and Heteroanion Dimers in Ultraclose Proximity within a Self-Assembled Hexacationic Cage. J. Am. Chem. Soc. 142, 20182. 10.1021/jacs.0c10253.
26. Sergeant, G. E., and Jolliffe, K. A. (2020). Anion recognition using a simple cyclic peptide. Supramol. Chem., 1. 10.1080/10610278.2020.1732377.
27. Zapata, F., Gonzalez, L., Bastida, A., Bautista, D., and Caballero, A. (2020). Formation of self-assembled supramolecular polymers by anti-electrostatic anion-anion and halogen bonding interactions. Chem. Commun. 56, 7084. 10.1039/d0cc02831b.
28. Wezenberg, S. J., Chen, L. J., Bos, J. E., Feringa, B. L., Howe, E. N. W., Wu, X., Siegler, M. A., and Gale, P. A. (2021). Photomodulation of Transmembrane Transport and Potential by Stiff-Stilbene Based Bis(thio)ureas. J. Am. Chem. Soc. 144, 331. 10.1021/jacs.1c10034.
29. Zhu, J., Sun, X. W., Yang, X., Yu, S. N., Liang, L., Chen, Y. Z., Zheng, X., Yu, M., Yan, L., Tang, J., et al. (2023). In Situ Photoisomerization of an Azobenzene-Based Triple Helicate with a Prolonged Thermal Relaxation Time. Angew. Chem., Int. Ed. 62, e202314510. 10.1002/anie.202314510.
30. Chen, Y., Kuvayskaya, A., Pink, M., Sellinger, A., and Flood, A. H. (2024). A library of vinyl phosphonate anions dimerize with cyanostars, form supramolecular polymers and undergo statistical sorting. Chem. Sci. 15, 389. 10.1039/d3sc03685e.
31. Sada, K., Tani, T., and Shinkai, S. (2006). Organic ammonium carboxylates as supramolecular building blocks: The role of ionic hydrogen bonding. Synlett, 2364. 10.1055/s-2006-951469.
32. Sessler, J. L., Gale, P. A., and Cho, W.-S. (2006). Anion Receptor Chemistry (Royal Society of Chemistry).
33. Sessler, J. L., Gross, D. E., Cho, W.-S., Lynch, V. M., Schmidtchen, F. P., Bates, G. W., Light, M. E., and Gale, P. A. (2006). Calix [4] pyrrole as a chloride anion receptor: solvent and countercation effects. J. Am. Chem. Soc. 128, 12281. 10.1021/ja064012h.
34. Hua, Y., Ramabhadran, R. O., Uduehi, E. O., Karty, J. A., Raghavachari, K., and Flood, A. H. (2011). Aromatic and aliphatic CH hydrogen bonds fight for chloride while competing alongside ion pairing within triazolophanes. Chem. Eur. J. 17, 312. 10.1002/chem.201002340.
35. Bowman-James, K. (2005). Alfred Werner revisited: the coordination chemistry of anions. Acc. Chem. Res. 38, 671. 10.1021/ar040071t.
36. Kang, S. O., Llinares, J. M., Day, V. W., and Bowman-James, K. (2010). Cryptand-like anion receptors. Chem. Soc. Rev. 39, 3980. 10.1039/c0cs00083c.
37. Bell, R. A., Christoph, G. G., Fronczek, F. R., and Marsh, R. E. (1975). The cation H13O6+: a short, symmetric hydrogen bond. Science 190, 151. 10.1126/science. 190.4210.151.
38. Metz, B., Rosalky, J. M., and Weiss, R. (1976). [3] Cryptates: X-ray crystal structures of the chloride and ammonium ion complexes of a spheroidal macrotricyclic ligand. Chem. Commun., 533b. 10.1039/C3976000533B.
39. Hossain, M. A., Liljegren, J. A., Powell, D., and Bowman-James, K. (2004). Anion binding with a tripodal amine. Inorg. Chem. 43, 3751. 10.1021/ic049762b.
40. Mascal, M., Yakovlev, I., Nikitin, E. B., and Fettinger, J. C. (2007). Fluoride-selective host based on anion-pi interactions, ion pairing, and hydrogen bonding: synthesis and fluoride-ion sandwich complex. Angew. Chem., Int. Ed. 46, 8782. 10.1002/anie.200704005.
41. Lin, W., Zhang, G., Zhu, X., Yu, P., Alimi, L. O., Moosa, B. A., Sessler, J. L., and Khashab, N. M. (2023). Caging the Hofmeister Effect by a Biomimetic Supramolecular Receptor. J. Am. Chem. Soc. 145, 12609. 10.1021/jacs.3c01849.
42. Zhao, W., Qiao, B., Tropp, J., Pink, M., Azoulay, J. D., and Flood, A. H. (2019). Linear Supramolecular Polymers Driven by Anion-Anion Dimerization of Difunctional Phosphonate Monomers Inside Cyanostar Macrocycles. J. Am. Chem. Soc. 141, 4980. 10.1021/jacs.9b00248.
43. Basit, H., Pal, A., Sen, S., and Bhattacharya, S. (2008). Two-component hydrogels comprising fatty acids and amines: structure, properties, and application as a template for the synthesis of metal nanoparticles. Chem. Eur. J. 14, 6534. 10.1002/chem.200800374.
44. Montero de Espinosa, L., Balog, S., and Weder, C. (2014). Isophthalic Acid-Pyridine H-Bonding: Simplicity in the Design of Mechanically Robust Phase-Segregated Supramolecular Polymers. ACS Macro. Lett. 3, 540. 10.1021/mz500293x.
45. Shmukler, L. E., Fedorova, I. V., Gruzdev, M. S., and Safonova, L. P. (2019). Triethylamine-Based Salts: Protic Ionic Liquids or Molecular Complexes? J. Phys. Chem. B 123, 10794. 10.1021/acs.jpcb.9b08032.
46. (a) For the nine 4° ammonium cations, we require 9 N-alkylations, which could be run in 2 days. Each would require purification largely by trial and error at possibly a success rate of 3 per day for 3 days. Thus, 9 chloride salts could be prepared in 2 days. The target organophosphate anions would need to be prepared by reaction of the acid with NaOH. The sodium and chloride salts could be used in a metathesis reaction facilitated by precipitation of NaCl to isolate the 4° ammonium salts of the phosphate of interest. Purification would be needed. The metathesis and purification would need to be conducted for each of the 72 target salts. Assuming the same rate of 3 purifications per day to total 24 days. The grand total would be 31 days or about 6 weeks. Given the typical factor of pi as the ratio between how long something takes versus how long you expect it to take, the time frame to make 72 salts might take 4-5 months. (b) Alternatively, after preparation of the nine 4° ammonium cations as BF4− or Cl− salts, they could be subject to metathesis with hydroxide to prepare the OH-versions. These would be amenable to acid-base reaction with the organophosphoric acids. Thus, after the alkylations (2 days), a reliable method for hydroxide metathesis would be needed, perhaps established over 2 weeks (10 days). Then, it could be repeated at 3 per day totaling 3 days. Generation of the target 72 salts would then proceed as per the acid-base reactions used with acids and 3° amines explored herein (2 days). The grand total would be 17 days factoring pi to generate the 72 within 3 months.
47. Jeffrey, G. A. (1997). An introduction to hydrogen bonding (Oxford university press New York).
48. Greenaway, R. L., Santolini, V., Bennison, M. J., Alston, B. M., Pugh, C. J., Little, M. A., Miklitz, M., Eden-Rump, E. G. B., Clowes, R., Shakil, A., et al. (2018). High-throughput discovery of organic cages and catenanes using computational screening fused with robotic synthesis. Nat. Commun. 9, 2849. 10.1038/s41467-018-05271-9.
49. Cui, P., McMahon, D. P., Spackman, P. R., Alston, B. M., Little, M. A., Day, G. M., and Cooper, A. I. (2019). Mining predicted crystal structure landscapes with high throughput crystallisation: old molecules, new insights. Chem. Sci. 10, 9988. 10.1039/c9sc02832c.
50. Ollerton, K., Greenaway, R. L., and Slater, A. G. (2021). Enabling technology for supramolecular chemistry. Front. Chem. 9, 774987. 10.3389/fchem.2021.774987.
51. Zhao, W., Yan, P., Li, B., Bahri, M., Liu, L., Zhou, X., Clowes, R., Browning, N. D., Wu, Y., Ward, J. W., and Cooper, A. I. (2022). Accelerated Synthesis and Discovery of Covalent Organic Framework Photocatalysts for Hydrogen Peroxide Production. J. Am. Chem. Soc. 144, 9902. 10.1021/jacs.2c02666.
52. Vijayakrishnan, S., Ward, J. W., and Cooper, A. I. (2022). Discovery of a Covalent Triazine Framework Photocatalyst for Visible-Light-Driven Chemical Synthesis using High-Throughput Screening. ACS Catal. 12, 10057. 10.1021/acscatal.2c02743.
53. Gattuso, G., Crisafulli, D., Milone, M., Mancuso, F., Pisagatti, I., Notti, A., and Parisi, M. F. (2022). Proton transfer mediated recognition of amines by ionizable macrocyclic receptors.

Chem. Commun. 58, 10743. 10.1039/d2cc04047f.
54. Trivedi, D. R., Ballabh, A., Dastidar, P., and Ganguly, B. (2004). Structure-property correlation of a new family of organogelators based on organic salts and their selective gelation of oil from oil/water mixtures. Chem. Eur. J. 10, 5311. 10.1002/chem.200400122.
55. Llinares, J. M., Powell, D., and Bowman-James, K. (2003). Ammonium based anion receptors. Coord. Chem. Rev. 240, 57. 10.1016/S0010-8545 (03) 00019-5.
56. Donald, J. E., Kulp, D. W., and DeGrado, W. F. (2011). Salt bridges: geometrically specific, designable interactions. Proteins 79, 898. 10.1002/prot.22927.
57. Blondeau, P., Segura, M., Perez-Fernandez, R., and de Mendoza, J. (2007). Molecular recognition of oxoanions based on guanidinium receptors. Chem. Soc. Rev. 36, 198. 10.1039/b603089k.
58. Dietrich, B., Fyles, T. M., Lehn, J.-M., Pease, L. G., and Fyles, D. L. (1978). Anion receptor molecules. Synthesis and some anion binding properties of macrocyclic guanidinium salts. Journal of the Chemical Society, Chemical Communications, 934-936.
59. Iida, H., Shimoyama, M., Furusho, Y., and Yashima, E. (2010). Double-Stranded Supramolecular Assembly through Salt Bridge Formation between Rigid and Flexible Amidine and Carboxylic Acid Strands. J. Org. Chem. 75, 417. 10.1021/jo902158v.
60. Jiang, H., Xie, L., Duan, Z., Lin, K., He, Q., Lynch, V. M., Sessler, J. L., and Wang, H. (2021). Fluorescent Supramolecular Organic Frameworks Constructed by Amidinium-Carboxylate Salt Bridges. Chem. Eur. J. 27, 15006-15012. 10.1002/chem.202102296.
61. Williams, N. J., Seipp, C. A., Brethome, F. M., Ma, Y.-Z., Ivanov, A. S., Bryantsev, V. S., Kidder, M. K., Martin, H. J., Holguin, E., Garrabrant, K. A., and Custelcean, R. (2019). CO2 Capture via Crystalline Hydrogen-Bonded Bicarbonate Dimers. Chem 5, 719. 10.1016/j.chempr.2018.12.025.
62. Manganaro, N., Pisagatti, I., Notti, A., Pappalardo, A., Patane, S., Micali, N., Villari, V., Parisi, M. F., and Gattuso, G. (2018). Ring/Chain Morphology Control in Overall-Neutral, Internally Ion-Paired Supramolecular Polymers. Chem. Eur. J. 24, 1097. 10.1002/chem.201703342.
63. Notti, A., Pisagatti, I., Nastasi, F., Patane, S., Parisi, M. F., and Gattuso, G. (2021). Stimuli-Responsive Internally Ion-Paired Supramolecular Polymer Based on a Bis-pillar[5]arene Dicarboxylic Acid Monomer. J. Org. Chem. 86, 1676. 10.1021/acs.joc.0c02501.
64. Fatila, E. M., Twum, E. B., Sengupta, A., Pink, M., Karty, J. A., Raghavachari, K., and Flood, A. H. (2016). Anions stabilize each other inside macrocyclic hosts. Angew. Chem., Int. Ed. 55, 14057. 10.1002/anie.201608118.
65. Zhao, W., Qiao, B., Chen, C. H., and Flood, A. H. (2017). High-Fidelity Multistate Switching with Anion-Anion and Acid-Anion Dimers of Organophosphates in Cyanostar Complexes. Angew. Chem., Int. Ed. 56, 13083. 10.1002/anie.201707869.
66. Mata, I., Alkorta, I., Molins, E., and Espinosa, E. (2012). Electrostatics at the origin of the stability of phosphate-phosphate complexes locked by hydrogen bonds. ChemPhysChem 13, 1421. 10.1002/cphc.201200068.
67. Weinhold, F., and Klein, R. A. (2014). Anti-electrostatic hydrogen bonds. Angew. Chem., Int. Ed. 53, 11214. 10.1002/anie.201405812.
68. Fatila, E. M., Pink, M., Twum, E. B., Karty, J. A., and Flood, A. H. (2018). Phosphate-phosphate oligomerization drives higher order co-assemblies with stacks of cyanostar macrocycles. Chem. Sci. 9, 2863. 10.1039/c7sc05290a.
69. Azofra, L. M., Elguero, J., and Alkorta, I. (2020). Stabilisation of dianion dimers trapped inside cyanostar macrocycles. Phys. Chem. Chem. Phys. 22, 11348. 10.1039/d0cp01321h.
70. Zhao, W., Flood, A. H., and White, N. G. (2020). Recognition and applications of anion-anion dimers based on anti-electrostatic hydrogen bonds (AEHBs). Chem. Soc. Rev. 49, 7893. 10.1039/d0cs00486c.
71. Belieres, J.-P., and Angell, C. A. (2007). Protic ionic liquids: preparation, characterization, and proton free energy level representation. J. Phys. Chem. B 111, 4926. 10.1021/jp067589u.
72. Stoimenovski, J., Izgorodina, E. I., and MacFarlane, D. R. (2010). Ionicity and proton transfer in protic ionic liquids. Phys. Chem. Chem. Phys. 12, 10341. 10.1039/c0cp00239a.
73. Amarasekara, A. S. (2016). Acidic ionic liquids. Chem. Rev. 116, 6133. 10.1021/acs.chemrev.5b00763.
74. Haskins, M. M., Lusi, M., and Zaworotko, M. J. (2022). Supramolecular Synthon Promiscuity in Phosphoric Acid-Dihydrogen Phosphate Ionic Cocrystals. Cryst. Growth Des. 22, 3333. 10.1021/acs.cgd.2c00150.
75. Aboudzadeh, M. A., Muñoz, M. E., Santamaría, A., Fernández-Berridi, M. J., Irusta, L., and Mecerreyes, D. (2012). Synthesis and Rheological Behavior of Supramolecular Ionic Networks Based on Citric Acid and Aliphatic Diamines. Macromolecules 45, 7599. 10.1021/ma300966m.
76. Lutovsky, G. A., Gockel, S. N., Bundesmann, M. W., Bagley, S. W., and Yoon, T. P. (2023). Iron-mediated modular decarboxylative cross-nucleophile coupling. Chem 9, 1610. 10.1016/j.chempr.2023.04.008.
77. McGrath, N. A., Brichacek, M., and Njardarson, J. T. (2010). A Graphical Journey of Innovative Organic Architectures That Have Improved Our Lives. J. Chem. Educ. 87, 1348. 10.1021/ed1003806.
78. Li, J., Huang, C.-Y., Han, J.-T., and Li, C.-J. (2021). Development of a Quinolinium/Cobaloxime Dual Photocatalytic System for Oxidative C-C Cross-Couplings via H2 Release. ACS Catal. 11, 14148. 10.1021/acscatal. 1c04073.
79. Zhao, W., Tropp, J., Qiao, B., Pink, M., Azoulay, J. D., and Flood, A. H. (2020). Tunable Adhesion from Stoichiometry-Controlled and Sequence-Defined Supramolecular Polymers Emerges Hierarchically from Cyanostar-Stabilized Anion-Anion Linkages. J. Am. Chem. Soc. 142, 2579. 10.1021/jacs.9b12645.
80. We found that the presence of a little water in acetonitrile lead to formation of the 2:2 species with the tertiary ammonium cation dissociated. Freshly opened bottles of acetonitrile were used in our screening studies.
81. McNally, J. S., Noll, B., Orme, C. J., and Wilson, A. D. (2015). Density functional theory analysis of the impact of steric interaction on the function of switchable polarity solvents. J. Phys. Chem. B 119, 6766. 10.1021/acs.jpcb.5b03167.
82. McNally, S., Wang, X. P., Hoffmann, C., and Wilson, A. D. (2017). Self-assembly of molecular ions via like-charge ion interactions and through-space defined organic domains. Chem. Commun. 53, 10934. 10.1039/c7cc06401b.
83. Williams, S. R., Borgerding, E. M., Layman, J. M., Wang, W., Winey, K. I., and Long, T. E. (2008). Synthesis and characterization of well-defined 12, 12-ammonium ionenes: Evaluating mechanical properties as a function of molecular weight. Macromolecules 41, 5216. 10.1021/ma8006152.
84. Ke, C., Yang, C., Mori, T., Wada, T., Liu, Y., and Inoue, Y. (2009). Catalytic enantiodifferentiating photocyclodimerization of 2-anthracenecarboxylic acid mediated by a non-sensitizing chiral metallosupramolecular host. Angew. Chem., Int. Ed. 48, 6675. 10.1002/anie.200902911.
85. Li, Y., Wang, Y., Huang, G., Ma, X., Zhou, K., and Gao, J. (2014). Chaotropic-anion-induced supramolecular self-assembly of ionic polymeric micelles. Angew. Chem., Int. Ed. 53, 8074. 10.1002/anie.201402525.
86. Palao, E., de la Moya, S., Agarrabeitia, A. R., Esnal, I., Banuelos, J., Lopez-Arbeloa, I., and Ortiz, M. J. (2014). Selective lateral lithiation of methyl BODIPYs: synthesis, photophysics, and electrochemistry of new meso derivatives. Org. Lett. 16, 4364. 10.1021/01501945v.
87. Zhu, H., Fan, J., Mu, H., Zhu, T., Zhang, Z., Du, J., and Peng, X. (2016). d-PET-controlled “off-on” Polarity-sensitive Probes for Reporting Local Hydrophilicity within Lysosomes. Sci. Rep. 6, 35627. 10.1038/srep35627.
88. Fa, S., Shi, T. H., Akama, S., Adachi, K., Wada, K., Tanaka, S., Oyama, N., Kato, K., Ohtani, S., Nagata, Y., et al. (2022). Real-time chirality transfer monitoring from statistically random to discrete homochiral nanotubes. Nat. Commun. 13, 7378. 10.1038/s41467-022-34827-Z.
89. Deng, Y., Frezel, A., Mehner, F., Friedel, P., and Gaitzsch, J. (2023). Amine-bearing cyclic ketene acetals for pH-responsive and degradable polyesters through radical ring-opening polymerisation. Polym. Chem. 14, 4275. 10.1039/d3py00929g.
90. Denmark, S. E., Gould, N. D., and Wolf, L. M. (2011). A Systematic Investigation of Quaternary Ammonium Ions as Asymmetric Phase-Transfer Catalysts. Synthesis of Catalyst Libraries and Evaluation of Catalyst Activity. J. Org. Chem. 76, 4260. 10.1021/jo2005445.
91. Despite modest differences in relative populations, reaction free energies are large after accounting for the stoichiometry of the formation reaction.

### Example 2

Modular non-covalent linkages allow the construction of supramolecular polymers with different topologies¹⁻⁵ and offer control over the resulting material properties.⁶⁻¹¹ In most cases, the synthesis of assemblies with sophisticated topologies requires the design of building blocks that rely heavily on multistep covalent synthesis.^{3, 8, 12-15} For example, to construct a network using metal-ligand coordination with a 2:1 stoichiometry, a multitopic ligand is required.¹⁶⁻¹⁸ If a specific topology is required, then geometry of the ligand needs to be carefully designed or the construction strategy needs to be carefully considered.^{8, 19-22} Instead of ligand design, in some cases, changes in topology can be achieved by varying the coordination center, which were seen in both cation- and anion-driven assemblies.^{7, 23-25} However, the versatile topologies mainly depend on the nature of coordination center, i.e., the metal cation^{7, 19, 22} or, the anion,²⁵⁻²⁶ providing less programmable space in terms of synthetic modification.

Cation-driven assembly has offered multitopic topologies, i.e., cage,²⁷⁻³⁰ linear polymer,³¹⁻³³ 2D^{9, 34-35} and 3D networks.^{15, 18} To data, anion-driven assembly only gives some of these³⁶⁻³⁹ due to the rarity of stable, modular assembly linkages and the difficulty to synthetically modify anionic guests and their receptor.

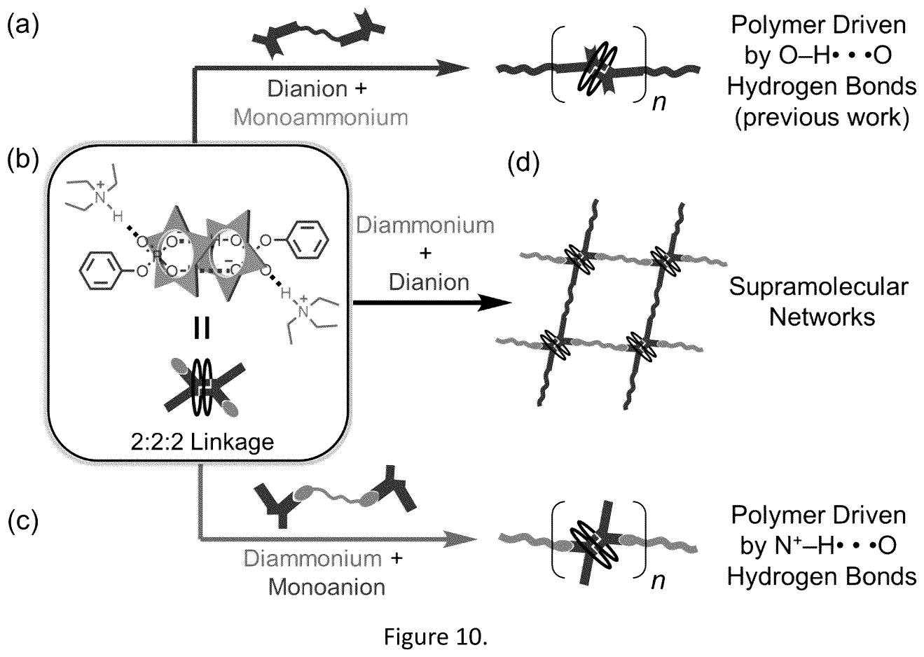

We previously reported the privileged 2:2:2 ternary assembly,⁴⁰ which consists of phosphate anions, tertiary ammonium cations and cyanostar macrocycles (FIG. 10b). The assembly is purely connected by multiple synergistic hydrogen bonds, i.e., O—H···O, N⁺—H···O and C—H···O. Previously, we have shown that the strong O—H···O hydrogen bonds between anions enable the supramolecular polymerization (FIG. 10a).^{36, 41-42} We envision that the N⁺—H···O salt bridge between cation and anion⁴⁰ is also strong enough to connect one assembly unit to another one, yielding a polymeric structure (FIG. 10c). More importantly, and different from the widely reported 2:1,^{4,6} and 1:1⁴³ linkage stoichiometry, the unique 2:2:2 linkage stoichiometry provides more choices in terms of design strategy to construct more sophisticated topology. For example, to get a network from 2:2 requests combination of diacid with a bis-macrocycle, which is a demanding synthesis. Using 2:2:2, we can simply replace bis-macrocycle by a diamine, for example, ditopic monomer with triethylamine end-groups (Et₂NCH₂CH₂—R—CH₂CH₂NEt₂) based on the simplest combination. Therefore, we hypothesize that combining both ditopic acids and ditopic amines can generate supramolecular networks supported by O—H···O, N⁺—H···O and C—H···O hydrogen bonds (FIG. 10d).

### Polymerization Driven by N⁺—H···O Salt Bridge

To test the supramolecular polymerization based on charge-assisted N⁺—H···O salt bridge, we show that ditopic tertiary ammonium cations will cross link the 2:2 cyanostar-anion assemblies. We mix the diamine with two equivalents of a mono-phosphoric acid. This ratio generates a monomer with two N⁺—H end-groups that, upon the addition of cyanostar, resulted in formation of a 2:2:2 assembly unit. These units are linked by ditopic ammonium through N⁺—H···O salt bridge (FIG. 10c).

Inspired by the formation of high-fidelity 2:2:2 assembly using triethylamine, phenyl phosphoric acid and cyanostar,⁴⁰ we start with the N¹,N¹,N¹²,N¹²-tetraethyldodecane-1,12-diamine (Et₂N—C₁₂H₂₄—NEt₂; NC₁₂N) due to its feasible synthesis⁴⁴ and similar steric profile compared to triethylamine. The diamine was synthesized by reacting the corresponding dibromide with excess diethylamine, which can be easily removed during solvent evaporation to give the pure diamine after washing. The diamine was mixed with 2 equivalents of phenyl phosphoric acid to yield fully protonated diammonium phosphate salt (HN⁺C₁₂NH⁺·(PhPO₄H⁻)₂). As expected, we observed the N⁺—H···O hydrogen bond signal at 12.7 ppm, indicating the formation of ion pairs with the phosphates at each end of the ditopic ammonium.

Formation of the 2:2:2 assembly unit was tested by adding aliquots of the diammonium bis-phosphate to cyanostar (1 mM, dichloromethane). Satisfyingly, upon addition of just 0.2 equivalents of the salt, the typical 8-line pattern for cyanostar dimerization was observed, along with characteristic downfield resonances at 10.1 and 15.3 ppm, indicating the formation of N⁺—H···O and O—H···O hydrogen bonds, respectively. These positions are identical to the ones observed in the ternary assembly of the model compound using triethylammonium in place of the ditopic thread.⁴⁰ In contrast to the ternary assembly, we observe incomplete formation of the assembly with signals for the free cyanostar and uncomplexed phosphate present at the equivalence point. These observations indicate the presence of an equilibrium between the ion pair and the 2:2:2 assembly unit, which was also observed in some of ternary assembly examples.⁴⁰

According to our previous study,³⁶ supramolecular polymerization driven by anion-anion dimerization is a concentration-driven mechanism. Therein, increasing the concentration of the ingredients converts shorter oligomers into extended 1D polymers.³⁶ We hypothesize that higher concentrations should also favor the formation of polymers relative to the ion paired monomers. Consistently, we observe a gradual increase in the signal for the 2:2:2 linkage unit concomitant with decreased signals from free cyanostar and uncomplexed phosphate. At 20 mM concentration, conversion is complete.

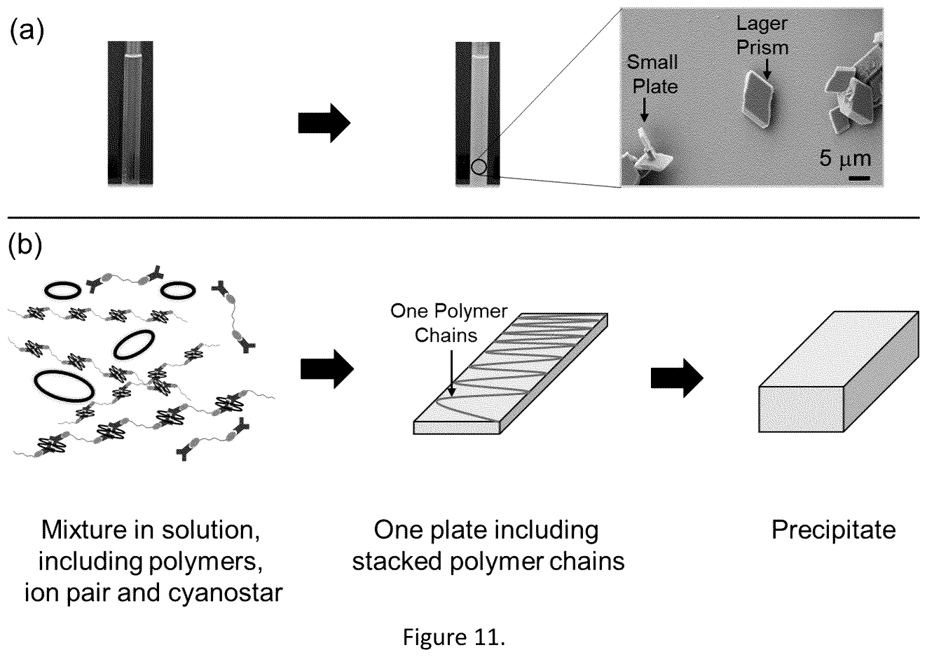

To confirm the assembled species is a polymer, the specific viscosity of the solution was measured as a function of concentration. A log-log plot shows a critical polymerization concentration (CPC) at around 3 mM consistent with a concentration-driven mechanism of polymerization.⁴⁵⁻⁴⁷ Unlike our previous anion-driven polymerization where precipitation was also observed beyond the critical polymerization concentration,³⁶ we observed precipitation across all concentrations approximately 1 hour after mixing the salt and cyanostar solutions (FIG. 11a). This outcome prevented use of diffusion NMR to accurately measure the size of the assemblies in solution. Therefore, all data, including 1D NMR and viscosity were collected using fresh solution without precipitates.

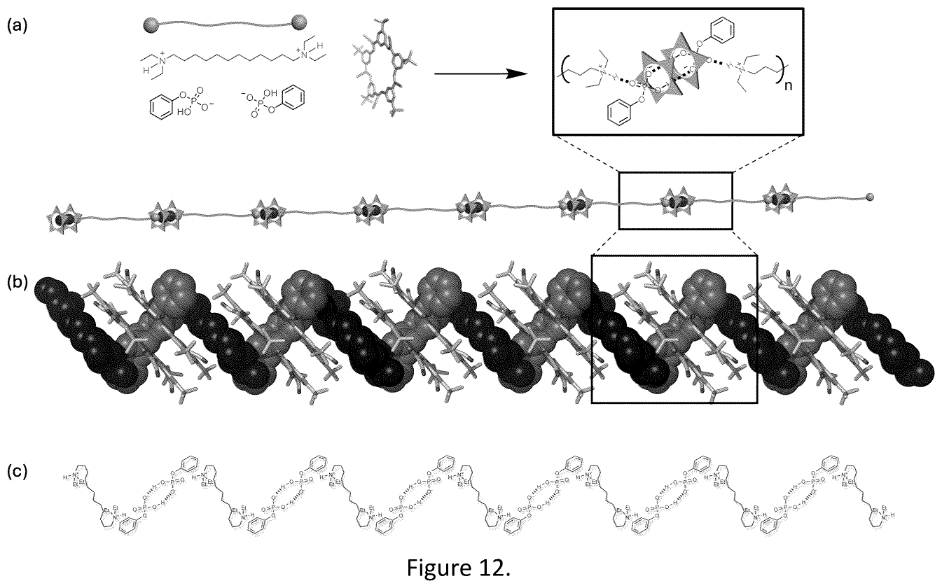

We were fortunate to grow crystals of sufficient quality to acquire a crystal structure (FIG. 12) of the 1D polymer. This structure confirms the linkage chemistries suggested from the NMR. Namely, the 2:2 stoichiometry of cyanostar: anion with the two phenyl-phosphate anions H-bonded to each other inside the p-stacked pair of macrocycles. In addition, we see the NH···O hydrogen bonding from the tertiary ammonium head groups and the phosphate oxygen atoms. Thus, the backbone of the polymer can be seen to be a 1D H-bonded chain of two monotopic anions and one ditopic ammonium dication.

By monitoring the NMR signal of freshly prepared polymer solution (1 mM), we found that the intensity of the proton signals corresponding to the assembly linkage gradually decreases and after 24 hours there is no significant linkage signal in solution while free cyanostar signals remain constant during the process. Therefore, we assume the precipitate is the polymer. To help gain more information of the precipitates arising from polymerization, we examined them using scanning electron microscope (SEM). The sample was prepared from the original 3-mM polymer suspension solution in dichloromethane. Briefly, 20 mL of the suspension was drop-cast onto silicon substrate without filtering the solution. The sample was slowly dried (˜60 min) in a sealed, dichloromethane-saturated chamber. This chamber allows some degree of solvent vapor annealing.

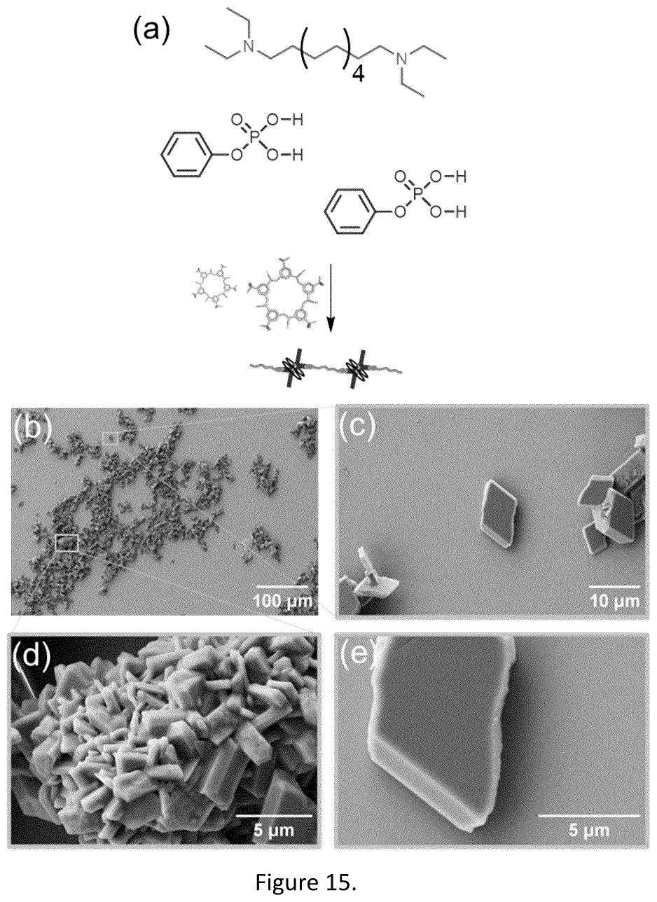

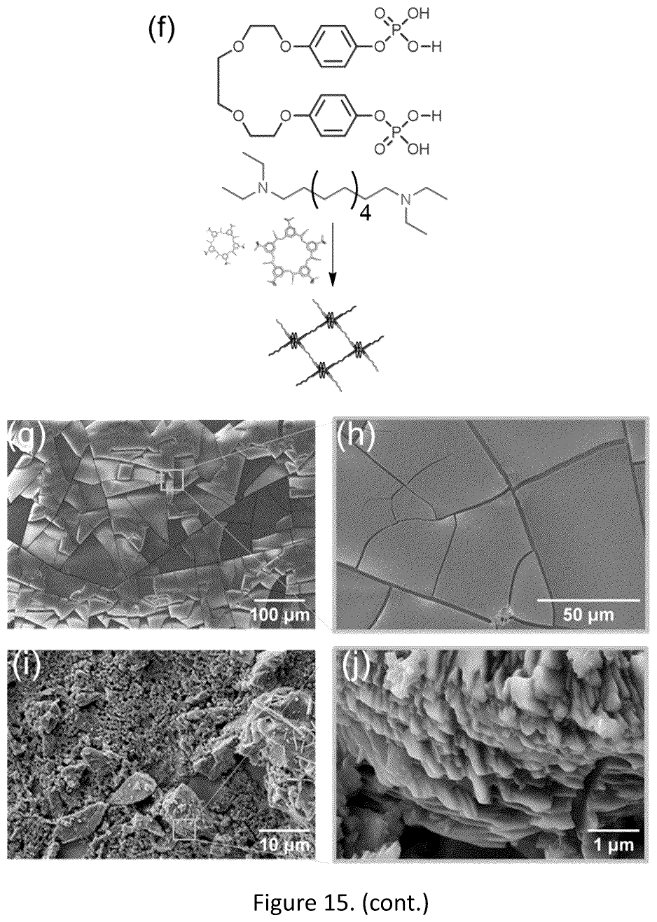

Surprisingly, many clusters of nearly uniform rhombic prism were observed under SEM (FIG. 15b). We confirmed that this morphology is solely due to assembly event by comparing these images with ones from the salt, cyanostar and ternary assembly linkage. By measuring a few discrete, intact rhombic prisms, they show a thickness of 1 mm, with the same 6×8 mm width and length (FIG. 15c and FIG. 15e). We also found a cluster of smaller rhombic prisms with 0.6 mm thickness (FIG. 15d). We hypothesize that the size of these prisms depends on the concentration and the precipitation duration. To test this hypothesis, we collected SEM images for precipitation from three different concentrations, i.e., 1, 3 and 10 mM. The SEM images show that higher concentrations favor formation of more objects, but the size difference of objects at different concentrations is not significant.

We also collected a series of SEM images at different time points for a 5 mM polymer solution. The solution was drop-cast onto the silicon substrate at different time points, e.g., 0, 20 and 60 min after mixing cyanostar and salt. The sample was quickly dried in an opened chamber such that further growth or annealing stopped immediately. The results show that after mixing cyanostar and salt, the assemblies are amorphous. These assemblies convert to larger clusters in the next 30 mins, and eventually become individual prism-like objects after 60 mins. This time-dependent assembly behavior is consistent with our observation, i.e., the precipitation happens in an approximately 1-hour duration after mixing different components and becomes stable in around 1 day. Interestingly, changing the diammonium monomer from N¹,N¹,N¹²,N¹²-tetraethyldodecane-1,12-diamine to the shorter N¹,N¹,N⁶,N⁶-tetramethylhexane-1,6-diamine results in a completely different rod-like morphology. It is worthy to note that this assembly precipitates immediately. Using NMR spectroscopy we observed no cyanostar dimer proton signals upon titrating the phosphate diammonium salt to cyanostar solution, but proton signal from free cyanostar gradually decreases when adding salt into cyanostar solution.

Combining the observation of relatively uniform micron-scale prisms, with the coexistence of NMR signals for the bound and unbound cyanostar, as well as precipitation over varying concentration, we assume that the polymeric assembly prefers to assemble hierarchically into larger objects over time. These objects possess poor solubility and thus precipitate, leaving the excess reactant, either cyanostar or ion pair in solution, which was seen in NMR spectra (FIG. 11).

Factors that Affect Supramolecular Polymerization

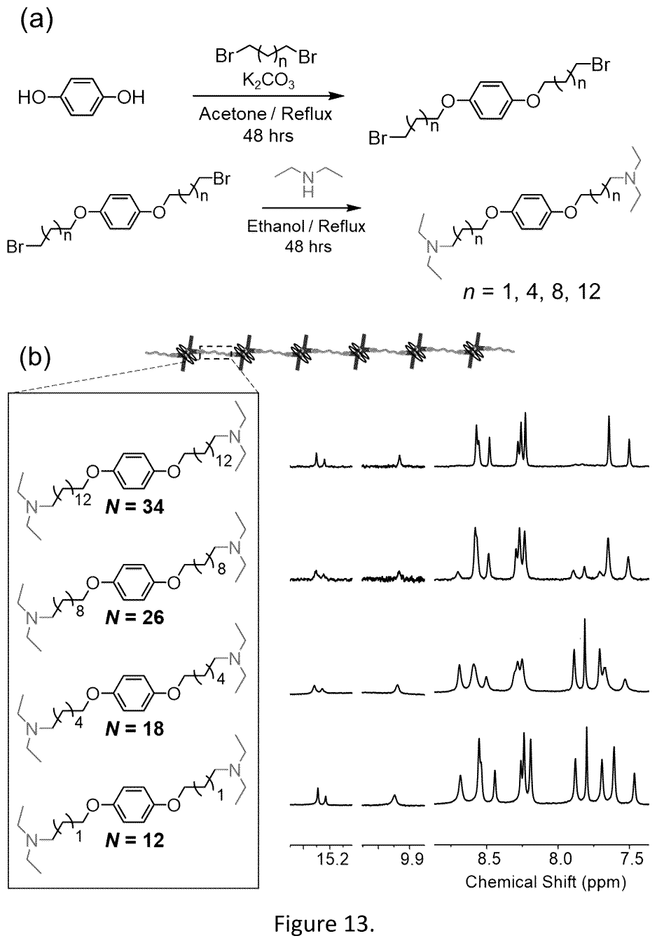

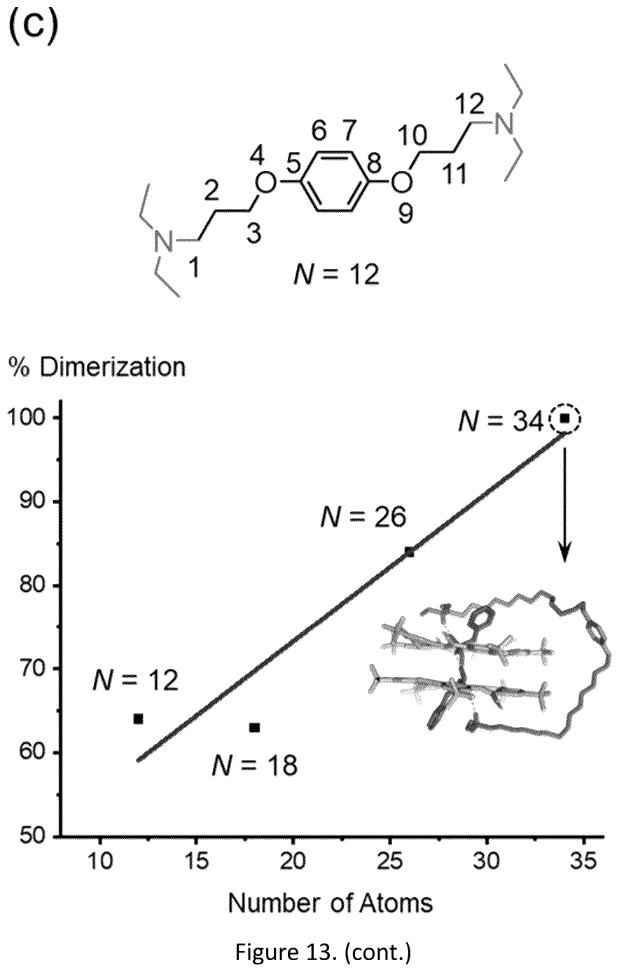

To better understand the origin of the incomplete assembly formation using diammonium phosphate salt (HN⁺C₁₂NH⁺·(PhPO₄H⁻)₂ and cyanostar at 1 mM. We tested the hypothesis that length of the linker between ammonium ends groups would impact assembly. The straightforward installation of tertiary amine end-groups allows the evaluation of a series of diamines with different lengths (FIG. 13a). For this purpose, we used a central diphenol that was substituted with chains of differing lengths and carrying bromides for alkylation with diethylamine. The shortest in this series (FIG. 13c) has a similar length to HN⁺—C₁₂—NH⁺ and it also shows incomplete formation of the 2:2:2 linkage.

A series of monomers were prepared by mixing these diamines with phenyl phosphoric acid, and subjected to supramolecular polymerization with cyanostar (1 mM) in dichloromethane (FIGS. 13b and c). We observe an increase in assembly formation with chain length. The longest diammonium with 34 atoms in length forms a pure 2:2:2 linkage. Instead of a linear polymer, modeling shows that the longest diamine also enables formation of self-associated cyclic species as a pseudo[3]catenane (FIG. 13c) that has a much smaller size than polymeric assembly. Fortunately, the assembly consisting of longest diammonium has good solubility in dichloromethane at low concentration, allowing us to obtain size information using diffusion NMR. 1 mM ternary assembly involving triethylamine, phenyl phosphoric acid, cyanostar and 1 mM polymer consisting of longest diamine, phenyl phosphoric acid and cyanostar show similar diffusion coefficient in dichloromethane, i.e., 5.9±0.3×10⁻¹⁰ m²·s⁻¹ for ternary assembly and 5.5±0.6×10⁻¹⁰ m²·s⁻¹ for polymer. These values are comparable to the one of the cyanostar dimer previously reported (5.63±0.03×10⁻¹⁰ m²·s⁻¹).⁴⁸ Therefore, the pure 2:2:2 linkage formed in the case of longest diammonium likely supports a self-associated cyclic species. This is also consistent with concentration-driven mechanisms revealed by polymerization system involving diammonium HN⁺—C₁₂—NH⁺. At a concentration below critical polymerization concentration, oligomers are the major species in solution.

We also tested the impact of random elements of the assembly system. According to our previous study,⁴⁰ increasing solvent polarity helps formation of 2:2:2 linkage, we observed the same effect for the polymerization driven by N⁺—H···O. Replacing the two ethyl groups by two smaller methyl groups does not improve the dimerization percentage. We also tried to modify the spacer between two amine end-groups by comparing alkyl chains, an aromatic moiety, and glycol chains of similar size (11-12 atoms between two nitrogen atoms), but did not observe a significant change, i.e., alkyl chain spacer gives 85% assembly, aromatic one is 64%, glycol one is 60%.

### Synthesis and NMR Characterization of 2D Supramolecular Networks

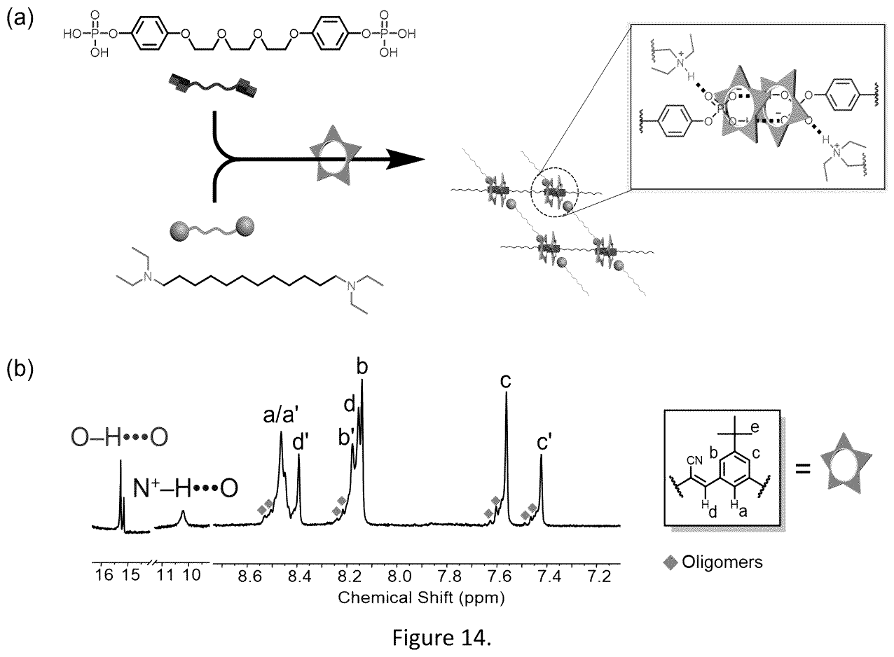

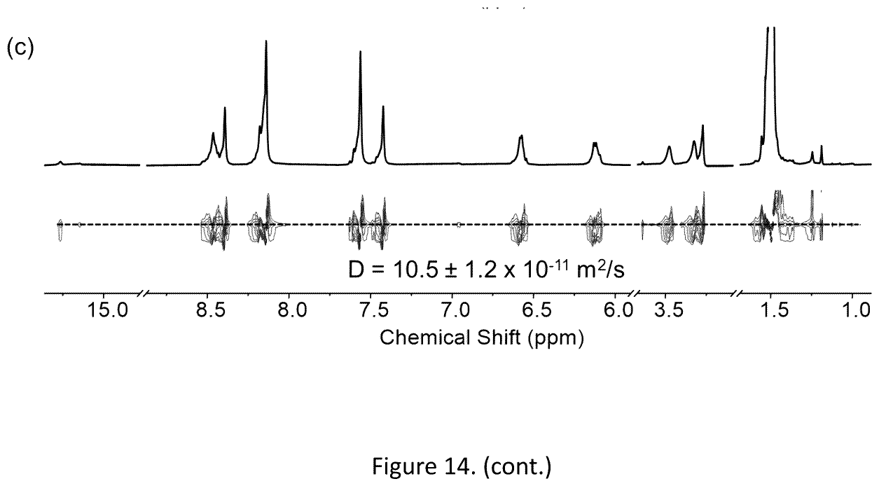

The successful polymerization driven by N⁺—H···O salt bridge using diammonium HN⁺—C₁₂—NH⁺ encouraged us to examine supramolecular network using the unique 2:2:2 stoichiometry.³⁶ The synthesis of a supramolecular network from low-molecular-weight building blocks relies on the stoichiometry of the recognition motif at the center of the non-covalent linkage. For example, considering a 2:1 stoichiometry typical for metal-coordination chemistry, the component present at the higher molar ratio can be converted into a multi-topic monomer to afford a network assembly.¹⁷ However, our novel 2:2:2 stoichiometry allows any one of the three components to be converted into a ditopic form for use as linear co-monomers. The simplest is diamine and di-acid where addition of cyanostar and the resulting 2:2:2 linkage has the potential to serve as a non-covalent crosslinker to assemble a supramolecular network (FIG. 14a).

To form a 2D network, we selected a diacid⁴¹ and diamine for which the values for the critical polymerization concentrations are already established independently as 1D polymers. The triethylene glycol bis(phenyl-p-phosphoric acid) and N¹,N¹,N¹²,N¹²-tetraethyldodecane-1,12-diamine (FIG. 14a) form 1D polymers in solvent and display CPCs values below 1 mM and at 3 mM, respectively. Accordingly, we examined the system at 10 mM cyanostar in dichloromethane to ensure polymerization.

When stoichiometric amounts of the diphosphate diammonium salt was mixed with cyanostar solution, an 8-line pattern appeared in the aromatic region with N⁺—H···O and O—H···O hydrogen bond signals shown at 10.2 and 15.2 ppm. No free cyanostar signals were observed (FIG. 14b). These signatures are indicative of complete formation of the 2:2:2 linkage. Surprisingly, when compared to the 1D polymerization using this same diamine (Et₂N—C₁₂—NEt₂), we did not observe precipitation of this supramolecular network in one week. Diffusion NMR shows that all peaks have the same diffusion coefficient (10.5±1.2×10⁻¹¹ m²/s, FIG. 14c). Based on the Stokes-Einstein equation for a spherical particle, the diameter of the network is around 10 nm, which is substantially larger than the size of the cyanostar (˜2 nm).

Interestingly, on the downfield side of the major peaks assigned to the cyanostar dimers involved in the network, we also observed a few correlated smaller peaks (FIG. 14b, diamonds). At a lower concentration (3 mM), instead of 8 major peaks in the aromatic region, we see 8 clusters of peaks, which suggest that these peaks are from smaller networks. This observation is consistent with the model of concentration-driven polymerization. Based on diffusion NMR at high concentration (10 mM), these smaller peaks have similar diffusion coefficients and are thus unlikely to be networks with different sizes. Alternatively, defeats in the networks or macrocycles on the edge of the networks might contribute to these small peaks. To evaluate these possibilities, we performed an annealing process in which the 10-mM network in dichloromethane was heated at 50° C. for 2 days. Therefore, small peaks appeared at 10 mM sample are likely the combination of defeats and macrocycles on the edge.

Inspired by the SEM results of the supramolecular polymer precipitation, we examined SEM images of the 3-mM supramolecular network solution and the 10-mM dried network as a suspension in dichloromethane. We saw a smooth layered morphology for the 3-mM solution sample with multiple cracks after being subjected to a relatively strong electron beam (FIGS. 15g and h). More interestingly, in the dried 10-mM sample (FIG. 15i), we observed a layer-by-layer morphology, with multiple layers stacked together to form a larger assembly (FIG. 15j). We assume that this type of structure is formed when the assembled networks are gradually concentrated upon solvent evaporation. By measuring one layer that is perpendicular to the surface, the thickness of this layer is around 120 nm. Further topological information of this network is probing by scanning tunneling microscope (STM) and single X-ray crystallography.

### Conclusion

In conclusion, we converted ammonium cations that are responsible for forming 2:2:2 assemblies with cyanostar-phosphate complexes into supramolecular polymers using ditopic monomers. The unique 2:2:2 stoichiometry allowed formation of supramolecular network by mixing ditopic co-monomers with cyanostar. For the first time, we observed these ion-driven assembly in the solid state by SEM imaging where different stages of the hierarchical assembly were captured. We believe this simple assembly will benefit from well-established amine and acid syntheses to allow sophisticated assemblies in both solution and solid phase to produce supramolecular materials. The successful application of our 2:2:2 ternary assembly inspires the further exploration of cations carrying functions.

### General Methods

Reagents were obtained from commercial suppliers and used as received unless otherwise indicated. The cyanostar macrocycle,¹ 1,4-bis(3-bromopropoxy)benzene,² 1,4-bis((6-bromohexyl)oxy)benzene,³ 1,4-bis((10-bromodecyl)oxy)benzene,⁴ tetraethylene glycol ditosylate and triethylene glycol bis(phenyl-p-phosphoric acid) 5 were synthesized according to the modified procedure. Column chromatography was performed on silica gel (160-200 mesh, Sorbent Technologies, USA). Thin-layer chromatography (TLC) was performed on pre-coated silica gel plates (0.25 mm thick) and observed under UV light. 1D Nuclear magnetic resonance (NMR) spectra and titrations were recorded on Varian Inova (500 and 600 MHz) and Bruker Avance Neospectrometers (500 MHz) at room temperature (298 K). Chemical shifts were referenced to residual solvent peaks. Scanning electron microscopy (SEM) images were taken on a Carl Zeiss Auriga 60 FIB-SEM operated at 1.50 kV. The Si substrates (<100> oriented, P/B type, University Wafer, Inc.) used for SEM image were sonicated in an isopropanol bath for 15 min and dried before use.

### Experimental Method for NMR Titration

In a forward titration, a solution of cyanostar macrocycle was prepared in an NMR tube sealed with a silicone septum and an initial spectrum was taken. A solution of phosphate ammonium salt was also prepared and added to the solution of cyanostar macrocycle with known quantities, the spectrum was recorded after each addition. In a reverse titration, a solution of phosphate ammonium salt was prepared in an NMR tube and a solution of cyanostar macrocycle was added to the solution of ammonium salt. All the spectra data were analyzed by using MestReNova software.

### Experimental Method for Viscosity

A series of solutions of cyanostar macrocycle with 0.5 equiv. of di-tertiary ammonium phosphate salt were prepared. Each sample was filtered and measured within 20 minutes after mixing cyanostar and salt to ensure no precipitation occurred. Every fresh sample was filtered with a syringe filter membrane before collection. Each sample was measured three times. For samples with a concentration below 1 mM, three samples were prepared, measured, and averaged for each concentration. Efflux time was collected and converted to specific viscosity.

### Experimental Method for Diffusion NMR

The diffusion coefficients were obtained based on the method of pulse gradient spin echo (PGSE) experiments. Aromatic regions were analyzed in this way to determine diffusion coefficients using Vnmrj's analysis. Average diffusion coefficients and errors were generated from multiple peaks used in analyses.

### SEM Sample Preparation

In a Teflon chamber, Si substrate was held by a plate holder, the chamber was first saturated with dichloromethane. Then 20 mL of sample solution/suspension was added on Si substrate by microsyringe. The chamber was covered by microscope slide till solvent is completely vaporized. The Si substrates was then subjected to sputter coating to coat a 3 nm Au/Pd layer.

### Synthesis and Characterization of Diamine

### N¹,N¹,N²,N²-tetraethyldodecane-1,12-diamine (Et₂NC₁₂NEt₂) 1⁶

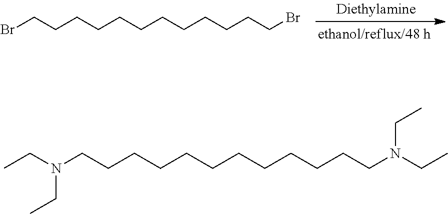 [SMILES: BrCCCCCCCCCCCCBr ; CCN(CC)CCCCCCCCCCCCN(CC)CC]

Scheme 1. Synthesis of N¹,N¹,N²,N²-tetraethyldodecane-1,12-diamine.

1,12-dibromododecane (328.1 mg, 1.00 mmol) was dissolved in ethanol (20.0 mL), excess diethylamine (4.0 mL, 40 equiv.) was added. The mixture was stirred under reflux for 48 h. The solvent was evaporated, and the residue was poured into a NaOH solution (1.00 M, 20 mL) and stirred. The solution was extracted with ethyl acetate (3×20 mL), and the organic phase was combined. After dried over anhydrous sodium sulfate, filtered and dried in vacuum to give 1 as brown liquid. The obtained compound was redissolved in EtOAc, filtered and dried in vacuum overnight. The yield of 1 was 97%. Dark brown solid was regenerated after the purification. ¹H NMR (600 MHz, CD₂Cl₂) δ (ppm): 2.46 (q, 8H), 2.39-2.32 (m, 4H), 1.39 (p, 4H), 1.27 (s, 16H), 0.97 (t, 12H). ¹³C NMR (126 MHz, CD₂Cl₂) δ (ppm): 47.01, 29.68, 29.66, 29.64, 27.65, 27.20, 11.69. HRMS (APCI) m/z: calcd, for C₂₀H₄₄N₂ at [M-H]+: 313.3577; found: 313.3582.

### 1,4-Bis[(14-bromotetradecyl)oxy]benzene, (BrC₁₄OPhOC₁₄Br) 2⁷

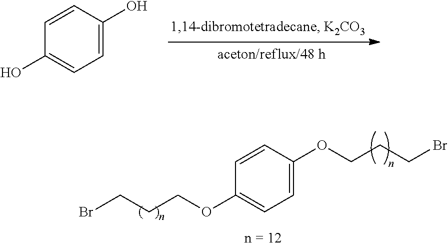

Scheme 2. Synthesis of 1,4-bis[(14-bromotetradecyl)oxy]benzene.

Hydroquinone (396 mg, 3.60 mmol) and 1,14-dibromodecane (5.13 g, 14.4 mmol) were dissolved in acetone (80 mL). Subsequently, anhydrous K₂CO₃ (1.51 g, 10.8 mmol) was reacted under reflux for 24 h. The reaction mixture was filtered, and the filtrate was evaporated under reduced pressure. The residue was chromatographed on a silica gel column using (CH₂C₁₂/Hexanes 1:4), then EtOAc as the mobile phase. Recrystallization from n-hexane gave product as a white solid (811 mg, 1.23 mmol, 34%). ¹H NMR (600 MHz, CD₂Cl₂) δ (ppm): 6.80 (s, 4H), 3.88 (t, 4H), 3.42 (t, 4H), 1.84 (p, 4H), 1.77-1.69 (m, 4H), 1.52 (s, 4H), 1.42 (d, 8H), 1.37-1.21 (m, 32H). ¹³C NMR (126 MHz, CD₂Cl₂) δ 153.22, 115.24, 68.60, 34.25, 32.91, 29.61, 29.59, 29.57, 29.53, 29.43, 29.40, 28.75, 28.16, 26.03. HRMS (EI) m/z: calcd, for C₃₄H₆₀O₂Br₂ at [M-H]+: 658.30; found: 658.2957.

### 3,3′-(1,4-phenylenebis(oxy))bis(N,N-diethylpropan-1-amine) (Et₂NC₃OPhOC₃NEt₂) 3

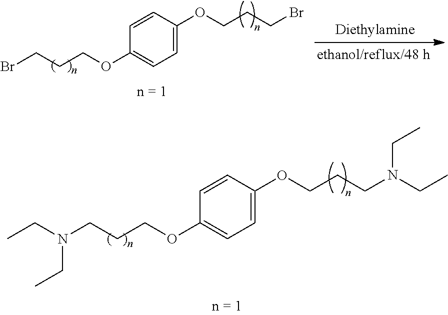

Scheme 3. Synthesis of 3,3′-(1,4-phenylenebis(oxy))bis(N,N-diethylpropan-1-amine)

1,4-bis(3-bromopropoxy)benzene (176 mg, 0.50 mmol) was dissolved in ethanol (10.0 mL), excess diethylamine (2.0 mL, 40 equiv.) was added. The mixture was stirred under reflux for 48 h. The solvent was evaporated, and the residue was poured into a NaOH solution (1.00 M, 10 mL) and stirred. The solution was extracted with ethyl acetate (3×10 mL), and the organic phase was combined. After dried over anhydrous sodium sulfate, filtered and dried in vacuum to give product as brown liquid. The obtained compound was redissolved in EtOAc, filtered and dried in vacuum overnight. The yield of 3 was 91%, ¹H NMR (600 MHz, CD₂Cl₂) δ (ppm): 6.81 (s, 4H), 3.94 (t, 4H), 2.58-2.53 (t, 4H), 2.50 (q, 8H), 1.89-1.81 (m, 4H), 0.99 (t, 12H). ¹³C NMR (126 MHz, CD₂Cl₂) δ (ppm): 153.67, 115.72, 67.36, 49.82, 47.41, 27.77, 12.19. HRMS (APCI) m/z: calcd, for C₂₀H₃₆N₂O₂ at [M-H]+: 337.2850; found: 337.2853.

### 6,6′-(1,4-phenylenebis(oxy))bis(N,N-diethylhexan-1-amine) (Et₂NC₆OPhOC₆NEt₂) 4

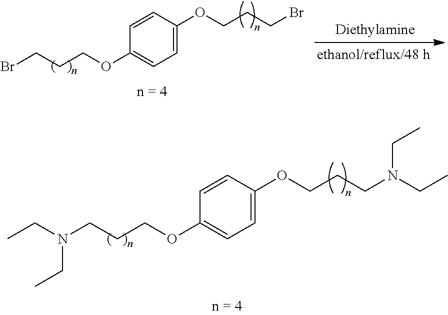

Scheme 4. Synthesis of 6,6′-(1,4-phenylenebis(oxy))bis(N,N-diethylhexan-1-amine).

1,4-bis((6-bromohexyl)oxy)benzene (218.0 mg, 0.50 mmol) was dissolved in ethanol (10.0 mL), excess diethylamine (2.0 mL, 40 equiv.) was added. The mixture was stirred under reflux for 48 h. The solvent was evaporated, and the residue was poured into a NaOH solution (1.00 M, 10 mL) and stirred. The solution was extracted with ethyl acetate (3×10 mL), and the organic phase was combined. After dried over anhydrous sodium sulfate, filtered and dried in vacuum to give 4 as brown solid. The obtained compound was redissolved in EtOAc, filtered and dried in vacuum overnight. The yield of 4 was 93%, ¹H NMR (500 MHz, CD₂Cl₂) ¿ (ppm): 6.77 (s, 4H), 3.86 (t, J=6.7 Hz, 4H), 2.44 (q, J=7.1 Hz, 8H), 2.37-2.32 (m, 4H), 1.75-1.67 (m, 4H), 1.48-1.36 (m, 9H), 1.35-1.26 (m, 4H), 0.95 (t, J=7.2 Hz, 12H). ¹³C NMR (126 MHz, CD₂C₁₂) δ (ppm): 153.62, 115.64, 68.93, 47.27, 29.82, 27.80, 27.55, 26.44, 12.09. HRMS (APCI) m/z: calcd, for C₂₆H₄₈N₂O₂ at [M-H]⁺: 421.3789; found: 421.3793.

### 10,10′-(1,4-phenylenebis(oxy))bis(N,N-diethyldecan-1-amine) (Et₂NC₁₀OPhOC₁₀NEt₂) 5

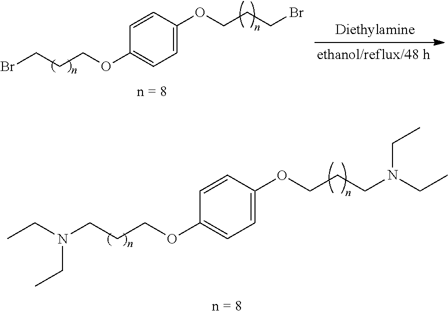

Scheme 5. Synthesis of 10,10′-(1,4-phenylenebis(oxy))bis(N,N-diethyldecan-1-amine).

1,4-bis((10-bromodecyl)oxy)benzene (274.2 mg, 0.50 mmol) was dissolved in ethanol (10.0 mL), excess diethylamine (2.0 mL, 40 equiv.) was added. The mixture was stirred under reflux for 48 h. The solvent was evaporated, and the residue was poured into a NaOH solution (1.00 M, 10 mL) and stirred. The solution was extracted with ethyl acetate (3×10 mL), and the organic phase was combined. After dried over anhydrous sodium sulfate, filtered and dried in vacuum to give 5 as brown solid. The obtained compound was redissolved in EtOAc, filtered and dried in vacuum overnight. The yield of 5 was 93%, ¹H NMR (600 MHz, CD₂Cl₂) δ (ppm): 6.79 (s, 4H), 3.88 (t, 4H), 2.46 (q, 8H), 2.35 (t, 4H), 1.73 (p, 4H), 1.47-1.21 (m, 28H), 0.97 (t, 12H). ¹³C NMR (126 MHz, CD₂Cl₂) δ (ppm): 153.68, 115.70, 69.05, 47.28, 30.08, 30.06, 30.04, 30.00, 29.84, 28.06, 27.54, 26.47, 12.05. HRMS (APCI) m/z: calcd, for C₃₄H₆₄N₂O₂ at [M-H]+: 533.5041; found: 533.5046.

### 10,10′-(1,4-phenylenebis(oxy))bis(N,N-dimethyldecan-1-amine) (Me₂NC₁₀OPhOC₁₀NMe₂) 6

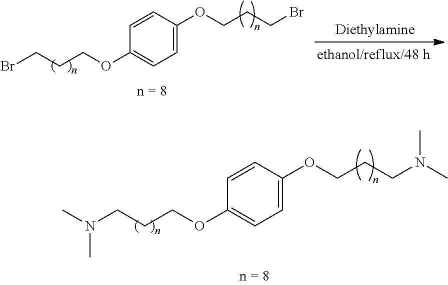

Scheme 6. Synthesis of 10,10′-(1,4-phenylenebis(oxy))bis(N,N-dimethyldecan-1-amine).

1,4-bis((10-bromodecyl)oxy)benzene (274.2 mg, 0.50 mmol) was dissolved in ethanol (10.0 mL), excess dimethylamine (11% ethanol included) (10.0 mL, 40 equiv.) was added. The mixture was stirred under reflux for 48 h. The solvent was evaporated, and the residue was poured into a NaOH solution (1.00 M, 10 mL) and stirred. The solution was extracted with ethyl acetate (3×10 mL), and the organic phase was combined. After dried over anhydrous sodium sulfate, filtered and dried in vacuum to give 6 as brown solid. The obtained compound was redissolved in EtOAc, filtered and dried in vacuum overnight. The yield of 6 was 93%, ¹H NMR (600 MHz, CD₂Cl₂) δ (ppm): 6.79 (s, 4H), 3.88 (t, 4H), 2.19 (t, 4H), 2.15 (s, 12H), 1.77-1.64 (m, 8H), 1.46-1.22 (m, 28H). ¹³C NMR (126 MHz, CD₂Cl₂) δ (ppm): 153.26, 115.26, 68.61, 59.89, 45.36, 29.67, 29.65, 29.62, 29.61, 29.45, 29.44, 27.78, 27.51, 26.07. HRMS (APCI) m/z: calcd. for C₃₀H₅₆N₂O₂ at [M-H]⁺: 477.4415; found: 477.4418.

### 14,14′-(1,4-phenylenebis(oxy))bis(N,N-diethyltetradecan-1-amine) (Et₂NC₁₄OPhOC₄NEt₂) 7

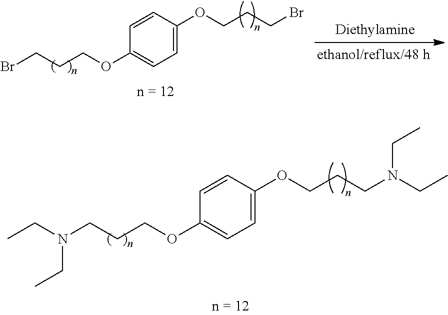

Scheme 7. Synthesis of 14,14′-(1,4-phenylenebis(oxy))bis(N,N-diethyltetradecan-1-amine).

1,4-Bis[(14-bromotetradecyl)oxy]benzene (330.3 mg, 0.50 mmol) was dissolved in ethanol (10.0 mL), excess diethylamine (2.0 mL, 40 equiv.) was added. The mixture was stirred under reflux for 48 h. The solvent was evaporated, and the residue was poured into a NaOH solution (1.00 M, 10 mL) and stirred. The solution was extracted with ethyl acetate (3×10 mL), and the organic phase was combined. After dried over anhydrous sodium sulfate, filtered and dried in vacuum to give 7 as brown solid. The obtained compound was redissolved in EtOAc, filtered and dried in vacuum overnight. The yield of 7 was 93%, ¹H NMR (600 MHz, CD₂Cl₂) δ (ppm): 6.79 (s, 4H), 3.88 (t, 4H), 2.47 (q, J=7.2 Hz, 8H), 2.39-2.34 (m, 4H), 1.76-1.68 (m, 4H), 1.48-1.37 (m, 8H), 1.27 (m, 36H), 0.98 (t, 12H). ¹³C NMR (126 MHz, CD₂Cl₂) δ (ppm): 153.22, 115.24, 68.60, 46.82, 29.67, 29.64, 29.59, 29.41, 27.63, 27.05, 26.03, 11.57. HRMS (APCI) m/z: calcd, for C₄₂H₈₀N₂O₂ at [M-H]+: 645.6293; found: 645.6296.

### 2,2′-((oxybis(ethane-2,1-diyl))bis(oxy))bis(N,N-diethylethan-1-amine) (Et₂NTEGNEt₂) 8

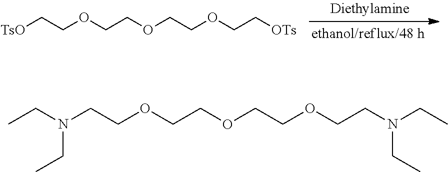 [SMILES: Cc1ccc(S(=O)(=O)OCCOCCOCCOCCOS(=O)(=O)c2ccc(C)cc2)cc1 ; CCN(CC)CCOCCOCCOCCN(CC)CC]

Scheme 8. Synthesis of 2,2′-((oxybis(ethane-2,1-diyl))bis(oxy))bis(N,N-diethylethan-1-amine).

Tetraethylene glycol ditosylate (514.7 mg, 1.00 mmol) was dissolved in ethanol (20.0 mL), excess diethylamine (4.0 mL, 40 equiv.) was added. The mixture was stirred under reflux for 48 h. The solvent was evaporated, and the residue was poured into a NaOH solution (1.00 M, 20 mL) and stirred. The solution was extracted with ethyl acetate (3×20 mL), and the organic phase was combined. After dried over anhydrous sodium sulfate, filtered and dried in vacuum to give 8 as brown liquid. The obtained compound was redissolved in EtOAc, filtered and dried in vacuum overnight. The yield of 8 was 87%. Dark brown solid was regenerated after the purification. ¹H NMR (600 MHz, CD₂Cl₂) δ (ppm): 3.59-3.53 (m, 8H), 3.49 (t, 4H), 2.60 (t, 4H), 2.52 (q, 8H), 0.98 (t, 12H). ¹³C NMR (126 MHz, CD₂Cl₂) δ 70.56, 70.38, 69.85, 47.52, 11.72, 11.67. HRMS (APCI) m/z: calcd, for C₁₆H₃₆N₂O₃ at [M-H]+: 305.2799; found: 305.2802.

### Standard Procedure of Converting Amine to Phosphate Ammonium Salt.

Small scale preparation: (<10 mg phosphoric acid)⁸ The ammonium phosphoric acid salt was formed by titrating the corresponding acid methanol solution with aliquots of tertiary amine dichloromethane solution until deprotonation was complete as verified by ¹H NMR spectroscopy based on the integration of amine's proton and proton of phenyl group from phosphoric acid. Then the solvent was evaporated, and the resulting salt was dried under vacuum for 1 day at room temperature before using for complexation. In some cases, i.e., Et₂NC₁₂NEt₂-bis(phenyl-p-phosphoric acid), Et₂NC₃OPhOC₃NEt₂-phosphoric acid, the solubility of the dry salt in dichloromethane was not very good, sonication was used and the equivalence point in salt-cyanostar titration was confirmed by proton integration between aromatic protons from cyanostar and aromatic protons from phenyl phosphate.

### REFERENCES FOR EXAMPLE 2

1. del Barrio, J.; Horton, P. N.; Lairez, D.; Lloyd, G. O.; Toprakcioglu, C.; Scherman, O. A., Photocontrol over cucurbit[8]uril complexes: stoichiometry and supramolecular polymers. J. Am. Chem. Soc. 2013, 135, 11760-3.
2. Rowland, M. J.; Appel, E. A.; Coulston, R. J.; Scherman, O. A., Dynamically crosslinked materials via recognition of amino acids by cucurbit[8]uril. J. Mater. Chem. B 2013, 1, 2904-2910.
3. Zhang, K. D.; Tian, J.; Hanifi, D.; Zhang, Y.; Sue, A. C.; Zhou, T. Y.; Zhang, L.; Zhao, X.; Liu, Y.; Li, Z. T., Toward a single-layer two-dimensional honeycomb supramolecular organic framework in water. J. Am. Chem. Soc. 2013, 135, 17913-17918.
4. Barrow, S. J.; Kasera, S.; Rowland, M. J.; del Barrio, J.; Scherman, O. A., Cucurbituril-Based Molecular Recognition. Chem. Rev. 2015, 115, 12320-406.
5. Xu, S. Q.; Zhang, X.; Nie, C. B.; Pang, Z. F.; Xu, X. N.; Zhao, X., The construction of a two-dimensional supramolecular organic framework with parallelogram pores and stepwise fluorescence enhancement. Chem. Commun. 2015, 51, 16417-16420.
6. Schubert, U. S.; Eschbaumer, C., Macromolecules containing bipyridine and terpyridine metal complexes: towards metallosupramolecular polymers. Angew. Chem., Int. Ed. 2002, 41, 2892-2926.
7. Beck, J. B.; Rowan, S. J., Multistimuli, multiresponsive metallo-supramolecular polymers. J. Am. Chem. Soc. 2003, 125, 13922-13923.
8. Lu, S.; Morrow, D. J.; Li, Z.; Guo, C.; Yu, X.; Wang, H.; Schultz, J. D.; O'Connor, J. P.; Jin, N.; Fang, F.; Wang, W.; Cui, R.; Chen, O.; Su, C.; Wasielewski, M. R.; Ma, X.; Li, X., Encapsulating Semiconductor Quantum Dots in Supramolecular Cages Enables Ultrafast Guest-Host Electron and Vibrational Energy Transfer. J. Am. Chem. Soc. 2023, 145, 5191-5202.
9. August, D. P.; Dryfe, R. A. W.; Haigh, S. J.; Kent, P. R. C.; Leigh, D. A.; Lemonnier, J. F.; Li, Z.; Muryn, C. A.; Palmer, L. I.; Song, Y.; Whitehead, G. F. S.; Young, R. J., Self-assembly of a layered two-dimensional molecularly woven fabric. Nature 2020, 588, 429-435.
10. Appel, E. A.; del Barrio, J.; Loh, X. J.; Scherman, O. A., Supramolecular polymeric hydrogels. Chem. Soc. Rev. 2012, 41, 6195-6214.
11. Kumpfer, J. R.; Rowan, S. J., Thermo-, photo-, and chemo-responsive shape-memory properties from photo-cross-linked metallo-supramolecular polymers. J. Am. Chem. Soc. 2011, 133, 12866-12874.
12. Sun, L.; Zhang, Z.; Leng, K.; Li, B.; Feng, C.; Huo, X., Can Supramolecular Polymers Become Another Material Choice for Polymer Flooding to Enhance Oil Recovery? Polymers 2022, 14.
13. Ronson, T. K.; Wang, Y.; Baldridge, K.; Siegel, J. S.; Nitschke, J. R., An S10-Symmetric 5-Fold Interlocked [2]Catenane. J. Am. Chem. Soc. 2020.
14. Wu, K.; Ronson, T. K.; Su, P.; Chen, Z.; Goh, L.; Heard, A. W.; Li, X.; Klautzsch, F.; Schalley, C. A.; Vinković, M.; Nitschke, J. R., Systematic construction of progressively larger capsules from a fivefold linking pyrrole-based subcomponent. Nat. Synth. 2023.
15. Ahmadi, M.; Seiffert, S., Direct Evidence of Heteroleptic Complexation in the Macroscopic Dynamics of Metallo-supramolecular Polymer Networks. Macromolecules 2021, 54, 7113-7124.
16. Winter, A.; Schubert, U. S., Synthesis and characterization of metallo-supramolecular polymers. Chem. Soc. Rev. 2016, 45, 5311-57.
17. Ueki, T.; Takasaki, Y.; Bundo, K.; Ueno, T.; Sakai, T.; Akagi, Y.; Yoshida, R., Autonomous viscosity oscillation via metallo-supramolecular terpyridine chemistry of branched poly (ethylene glycol) driven by the Belousov-Zhabotinsky reaction. Soft Matt. 2014, 10, 1349-1355.
18. Wang, Y.; Gu, Y.; Keeler, E. G.; Park, J. V.; Griffin, R. G.; Johnson, J. A., Star PolyMOCs with Diverse Structures, Dynamics, and Functions by Three-Component Assembly. Angew. Chem., Int. Ed. 2017, 56, 188-192.
19. Wang, H.; Li, Y.; Li, N.; Filosa, A.; Li, X., Increasing the size and complexity of discrete 2D metallosupramolecules. Nat. Rev. Mater. 2020, 6, 145-167.
20. Schultz, A.; Li, X.; Barkakaty, B.; Moorefield, C. N.; Wesdemiotis, C.; Newkome, G. R., Stoichiometric self-assembly of isomeric, shape-persistent, supramacromolecular bowtie and butterfly structures. J. Am. Chem. Soc. 2012, 134, 7672-7675.
21. Chakraborty, S.; Newkome, G. R., Terpyridine-based metallosupramolecular constructs: tailored monomers to precise 2D-motifs and 3D-metallocages. Chem. Soc. Rev. 2018, 47, 3991-4016.
22. Chakrabarty, R.; Mukherjee, P. S.; Stang, P. J., Supramolecular coordination: self-assembly of finite two- and three-dimensional ensembles. Chem. Rev. 2011, 111, 6810-6918.
23. Gavette, J. V.; Evoniuk, C. J.; Zakharov, L. N.; Carnes, M. E.; Haley, M. M.; Johnson, D. W., Exploring Anion-Induced Conformational Flexibility and Molecular Switching in a Series of Heteroaryl-Urea Receptors. Chem. Sci. 2014, 5, 2899-2905.
24. Gale, P. A., Anion coordination and anion-directed assembly: highlights from 1997 and 1998. Coord. Chem. Rev. 2000, 199, 181-233.
25. Bowman-James, K., Alfred Werner revisited: the coordination chemistry of anions. Acc. Chem. Res. 2005, 38, 671-678.
26. Liang, L.; Zhao, W.; Yang, X. J.; Wu, B., Anion-Coordination-Driven Assembly. Acc. Chem. Res. 2022, 55, 3218-3229.
27. Fujita, D.; Ueda, Y.; Sato, S.; Mizuno, N.; Kumasaka, T.; Fujita, M., Self-assembly of tetravalent Goldberg polyhedra from 144 small components. Nature 2016, 540, 563-566.
28. Saha, S.; Regeni, I.; Clever, G. H., Structure relationships between bis-monodentate ligands and coordination driven self-assemblies. Coord. Chem. Rev. 2018, 374, 1-14.
29. Rizzuto, F. J.; Nitschke, J. R., Narcissistic, Integrative, and Kinetic Self-Sorting within a System of Coordination Cages. J. Am. Chem. Soc. 2020.
30. Jiang, W.-L.; Huang, B.; Zhao, X.-L.; Shi, X.; Yang, H.-B., Strong halide anion binding within the cavity of a conformation-adaptive phenazine-based Pd2L4 cage. Chem 2023, 9, 2655-2668.
31. Fox, J. D.; Rowan, S. J., Supramolecular Polymerizations and Main-Chain Supramolecular Polymers. Macromolecules 2009, 42, 6823-6835.
32. Chiper, M.; Hoogenboom, R.; Schubert, U. S., Toward main chain metallo-terpyridyl supramolecular polymers: “the metal does the trick”. Macromol. Rapid. Commun. 2009, 30, 565-578.
33. Weng, W.; Li, Z.; Jamieson, A. M.; Rowan, S. J., Control of gel morphology and properties of a class of metallo-supramolecular polymers by good/poor solvent environments. Macromolecules 2009, 42, 236-246.
34. Yan, X.; Cook, T. R.; Pollock, J. B.; Wei, P.; Zhang, Y.; Yu, Y.; Huang, F.; Stang, P. J., Responsive supramolecular polymer metallogel constructed by orthogonal coordination-driven self-assembly and host/guest interactions. J. Am. Chem. Soc. 2014, 136, 4460-4463.
35. Su, F.; Zhang, S.; Chen, Z.; Zhang, Z.; Li, Z.; Lu, S.; Zhang, M.; Fang, F.; Kang, S.; Guo, C.; Su, C.; Yu, X.; Wang, H.; Li, X., Precise Synthesis of Concentric Ring, Helicoid, and Ladder Metallo-Polymers with Chevron-Shaped Monomers. J. Am. Chem. Soc. 2022, 144, 16559-16571.
36. Zhao, W.; Qiao, B.; Tropp, J.; Pink, M.; Azoulay, J. D.; Flood, A. H., Linear Supramolecular Polymers Driven by Anion-Anion Dimerization of Difunctional Phosphonate Monomers Inside Cyanostar Macrocycles. J. Am. Chem. Soc. 2019, 141, 4980-4989.
37. Bai, X.; Jia, C.; Zhao, Y.; Yang, D.; Wang, S. C.; Li, A.; Chan, Y. T.; Wang, Y. Y.; Yang, X. J.; Wu, B., Peripheral Templation-Modulated Interconversion between an A(4) L(6) Tetrahedral Anion Cage and A(2) L(3) Triple Helicate with Guest Capture/Release. Angew. Chem., Int. Ed. 2018, 57, 1851-1855.
38. Ma, F.; Qiao, X.; Zuo, W.; Tao, Y.; Li, A.; Luo, Z.; Liu, Y.; Liu, X.; Wang, X.; Sun, W.; Jia, C., Less is More: A Shortcut for Anionocages Design Based on (RPO(3) (2-))-Monourea Coordination. Angew. Chem., Int. Ed. 2022, 61, e202210478.
39. Zhao, X.; Wang, H.; Li, B.; Zhang, W.; Li, X.; Zhao, W.; Janiak, C.; Heard, A. W.; Yang, X. J.; Wu, B., A Hydrogen-Bonded Ravel Assembled by Anion Coordination. Angew. Chem., Int. Ed. 2022, 61, e202115042.
40. Chen, Y.; Kuvayskaya, A.; Pink, M.; Carta, V.; Sellinger, A.; Flood, A. H., Diversifying Hierarchical Ionic Assembly by Docking Cations to Anions as Salt Bridges. Chem 2024, 10, 3582-3594.
41. Zhao, W.; Tropp, J.; Qiao, B.; Pink, M.; Azoulay, J. D.; Flood, A. H., Tunable Adhesion from Stoichiometry-Controlled and Sequence-Defined Supramolecular Polymers Emerges Hierarchically from Cyanostar-Stabilized Anion-Anion Linkages. J. Am. Chem. Soc. 2020, 142, 2579-2591.
42. Chen, Y.; Kuvayskaya, A.; Pink, M.; Sellinger, A.; Flood, A. H., A library of vinyl phosphonate anions dimerize with cyanostars, form supramolecular polymers and undergo statistical sorting. Chem. Sci. 2024, 15, 389-398.
43. Ligthart, G. B. W. L.; Ohkawa, H.; Sijbesma, R. P.; Meijer, E. W., Complementary Quadruple Hydrogen Bonding in Supramolecular Copolymers. J. Am. Chem. Soc. 2005, 127, 810-811.
44. Jie, K.; Zhou, Y.; Shi, B.; Yao, Y., A Cu (2+) specific metallohydrogel: preparation, multi-responsiveness and pillar[5]arene-induced morphology transformation. Chem. Commun. 2015, 51, 8461-4.
45. Huang, F.; Nagvekar, D. S.; Zhou, X.; Gibson, H. W., Formation of a linear supramolecular polymer by self-assembly of two homoditopic monomers based on the bis(m-phenylene)-32-crown-10/paraquat recognition motif. Macromolecules 2007, 40, 3561-3567.
46. Wang, F.; Zheng, B.; Zhu, K.; Zhou, Q.; Zhai, C.; Li, S.; Li, N.; Huang, F., Formation of linear main-chain polypseudorotaxanes with supramolecular polymer backbones via two self-sorting host-guest recognition motifs. Chem. Commun. 2009, 4375-7.
47. Yang, L.; Tan, X.; Wang, Z.; Zhang, X., Supramolecular Polymers: Historical Development, Preparation, Characterization, and Functions. Chem. Rev. 2015, 115, 7196-239.
48. Zhao, W.; Qiao, B.; Chen, C. H.; Flood, A. H., High-Fidelity Multistate Switching with Anion-Anion and Acid-Anion Dimers of Organophosphates in Cyanostar Complexes. Angew. Chem., Int. Ed. 2017, 56, 13083-13087.

1. A supramolecular material comprising a 2:2:2 assembly, the 2:2:2 assembly comprising
a pair of macrocycles,
two anionic species dimerized within the pair of macrocycles, each anionic species is a conjugate base of an organophosphoric acid moiety or a conjugate base of an organophosphonic acid moiety,
and two cationic species, wherein each of the cationic species comprise a protonated nitrogen and each of the cationic species are hydrogen bonded by a N⁺—H···O salt bridge to an oxygen of the anionic species.

2. The supramolecular material of claim 1, wherein each conjugate acid of each of the two anionic species and each conjugate base of the two cationic species differ in pKa by greater than 3 units.

3. The supramolecular material of claim 1, wherein one or both of the cationic species is a tertiary ammonium moiety.

4. The supramolecular material of claim 3, wherein the cationic species is derived from a compound selected from the group consisting of: N,N-diisopropylpentan-3-amine, tribenzylamine, N-cyclohexyl-N-methylcyclohexanamine, triisopentylamine, 4-(tert-butyl)pyridine, 4-methylquinoline, 9-phenylacridine, 4-(trifluoromethyl)pyridine, N1,N1,N6,N6-tetramethylhexane-1,6-diamine,N1,N1,N²,N2-tetraethyldodecane-1,12-diamine, 3,3′-(1,4-phenylenebis(oxy))bis(N,N-diethylpropan-1-amine), 6,6′-(1,4-phenylenebis(oxy))bis(N,N-diethylhexan-1-amine), 10,10′-(1,4-phenylenebis(oxy))bis(N,N-diethyldecan-1-amine), 10,10′-(1,4-phenylenebis(oxy))bis(N,N-dimethyldecan-1-amine), 14,14′-(1,4-phenylenebis(oxy))bis(N,N-diethyltetradecan-1-amine), and 2,2′-((oxybis(ethane-2,1-diyl))bis(oxy))bis(N,N-diethylethan-1-amine).

5. The supramolecular material of claim 1, wherein one or both of the cationic species is a protonated nitrogen-containing heteroaryl moiety.

6. The supramolecular material of claim 5, wherein cationic species is a pyridinium moiety, a quinolinium moiety, an acridinium moiety, or an imidazolium moiety.

7. The supramolecular material of claim 1, wherein each of the anionic species is derived from a compound selected from the group consisting of:

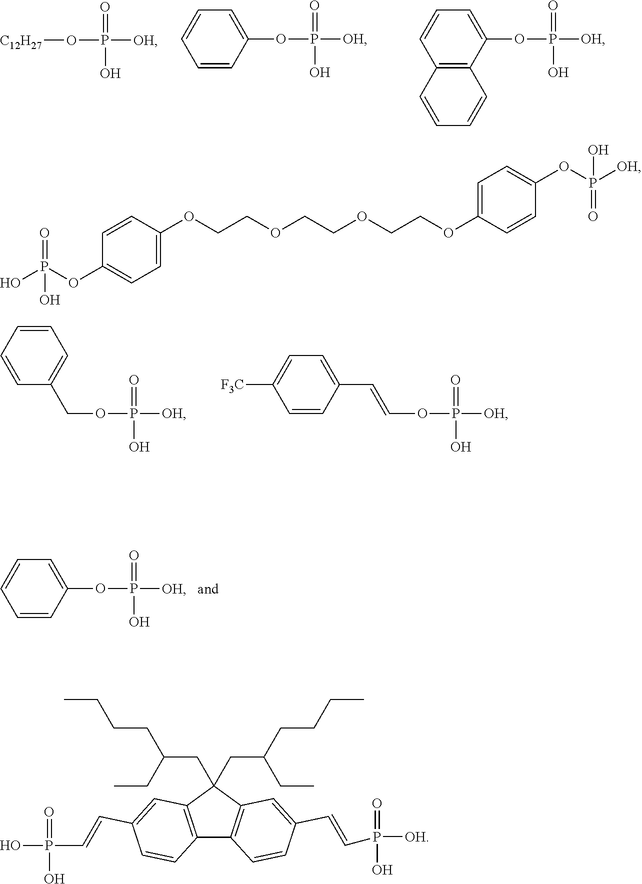

9. The supramolecular material of claim 1, wherein each macrocycle is a polycyanostilbene.

10. The supramolecular material of claim 9, wherein each macrocycle is

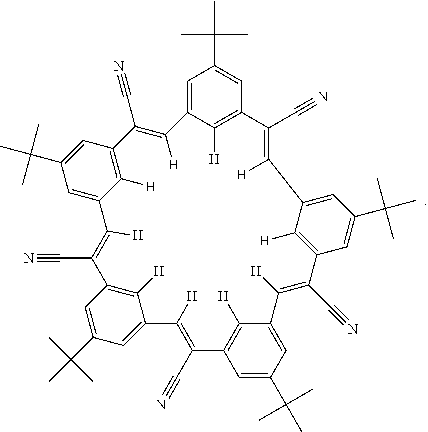 [SMILES: CC(C)(C)c1cc2cc(c1)/C(C#N)=C/c1cc(cc(C(C)(C)C)c1)/C(C#N)=C/c1cc(cc(C(C)(C)C)c1)/C(C#N)=C/c1cc(cc(C(C)(C)C)c1)/C(C#N)=C/c1cc(cc(C(C)(C)C)c1)/C(C#N)=C/2]

11. A supramolecular polymer comprising a plurality of 2:2:2 assemblies according to claim 1.

12. The supramolecular polymer of claim 11, wherein the supramolecular polymer comprises a plurality of compounds having at least one tertiary ammonium moiety or at least one protonated nitrogen-containing heteroaryl moiety.

13. The supramolecular polymer of claim 12, wherein the plurality of compounds comprise at least two tertiary ammonium moieties.

14. The supramolecular polymer of claim 13, wherein the supramolecular polymer is a linear polymer.

15. The supramolecular polymer of claim 13, wherein the supramolecular polymer is a network polymer.

14. The supramolecular polymer of claim 11, wherein the supramolecular polymer comprises a plurality of compounds having at least one phosphate moiety or at least one phosphonate moiety.

15. The supramolecular polymer of claim 14, wherein the supramolecular polymer is a linear polymer.

16. The supramolecular polymer of claim 11, wherein the supramolecular polymer comprises a plurality of compounds having at least two phosphate moieties or at least two phosphonate moieties.

17. The supramolecular polymer of claim 16, wherein the supramolecular polymer is a network polymer.

18. A method for preparing a material, the method comprising:
mixing macrocycles, first compounds having at least one tertiary amine moiety or at least one nitrogen-containing heteroaryl moiety, and second compounds having at least one phosphoric acid moiety or at least one phosphonic acid moiety,
wherein the mixing is performed under conditions suitable for proton transfer from second compounds to first compounds and formation of N⁺—H···O salt bridges between first compounds and second compounds.

19. The method of claim 18, wherein the macrocycles are added to a mixture of first compounds and second compounds.

20. The method of claim 18, wherein the first compounds comprise at least two tertiary ammonium moieties.
